(19) United States
(12) Patent Application Publication
(10) Pub. No.: US 2024/0125776 A1
(43) Pub. Date: Apr. 18, 2024

(54) MAGNETIC MICROCARRIERS FOR IMAGE DIFFERENTIATED MULTIPLEX ASSAYS

(71) Applicant: Plexbio Co., Ltd., Taipei City (TW)

(72) Inventors: Dean TSAO, Hillsborough, CA (US); Chin-Shiou HUANG, Santa Clara, CA (US); Cheng-Tse LIN, Taipei City (TW); Chien-Te WU, Taipei City (TW); FengKan LU, New Taipei City (TW)

(73) Assignee: Plexbio Co., Ltd., Taipei City (TW)

(21) Appl. No.: 18/516,803

(22) Filed: Nov. 21, 2023

Related U.S. Application Data
(63) Continuation of application No. PCT/US2022/031049, filed on May 26, 2022.

(51) Int. Cl. G01N 33/543; G01N 21/64; G01N 33/531

(52) U.S. Cl. CPC G01N 33/54326; G01N 21/6458; G01N 33/531; G01N 33/54346; G01N 2446/10

(57) ABSTRACT

Provided herein are encoded microcarriers for analyte detection in multiplex assays. The microcarriers are encoded with an analog code for identification and comprise a capture agent for analyte detection and a substantially transparent magnetic polymer. The analog code is generated by a two-dimensional shape of a substantially non-transparent layer. Also provided are methods of making the encoded microcarriers disclosed herein. Further provided are methods and kits for conducting a multiplex assay using the microcarriers described herein.

### CROSS-REFERENCE TO RELATED APPLICATION

This application claims the priority benefit of U.S. Provisional Patent Application No. 63/193,338 filed May 26, 2021, the entire content of which is incorporated herein in its entirety.

### FIELD

Provided herein are encoded microcarriers with a substantially transparent magnetic polymer layer useful, e.g., for analyte detection in multiplex assays, as well as methods of making and using the same and kits related thereto. In some embodiments, the microcarriers comprise a capture agent for capturing an analyte and are encoded with an analog code made from the two-dimensional shape of a non-transparent layer.

### BACKGROUND

Immunological and molecular diagnostic assays play a critical role both in the research and clinical fields. Often it is necessary to perform assays for a panel of multiple targets to gain a meaningful or bird's-eye view of results to facilitate research or clinical decision-making. This is particularly true in the era of genomics and proteomics, where an abundance of genetic markers and/or biomarkers are thought to influence or be predictive of particular disease states. In theory, assays of multiple targets can be accomplished by testing each target separately in parallel or sequentially in different reaction vessels (i.e., multiple singleplexing). However, not only are assays adopting a singleplexing strategy often cumbersome, but they also typically required large sample volumes, especially when the targets to be analyzed are large in number.

A multiplex assay simultaneously measures multiple analytes (two or more) in a single assay. Multiplex assays are commonly used in high-throughput screening settings, where many specimens can be analyzed at once. It is the ability to assay many analytes simultaneously and many specimens in parallel that is the hallmark of multiplex assays and is the reason that such assays have become a powerful tool in fields ranging from drug discovery to functional genomics to clinical diagnostics. In contrast to singleplexing, by combining all targets in the same reaction vessel, the assay is much less cumbersome and much easier to perform, since only one reaction vessel is handled per sample. The required test samples can thus be dramatically reduced in volume, which is especially important when samples (e.g., tumor tissues, cerebral spinal fluid, or bone marrow) are difficult and/or invasive to retrieve in large quantities. Equally important is the fact that the reagent cost can be decreased and assay throughput increased drastically.

Many assays of complex macromolecule samples are composed of two steps. In the first step, agents capable of specifically capturing the target macromolecules are attached to a solid phase surface. These immobilized molecules may be used to capture the target macromolecules from a complex sample by various means, such as hybridization (e.g., in DNA, RNA based assays) or antigen-antibody interactions (in immunoassays). In the second step, detection molecules are incubated with and bind to the complex of capture molecule and the target, emitting signals such as fluorescence or other electromagnetic signals. The amount of the target is then quantified by the intensity of those signals.

Multiplex assays may be carried out by utilizing multiple capture agents, each specific for a different target macromolecule. In chip-based array multiplex assays, each type of capture agent is attached to a pre-defined position on the chip. The amount of multiplex targets in a complex sample is determined by measuring the signal of the detection molecule at each position corresponding to a type of capture agent. In suspension array multiplex assays, microparticles or microcarriers are suspended in the assay solution. These microparticles or microcarriers contain an identification element, which may be embedded, printed, or otherwise generated by one or more elements of the microparticle/microcarrier. Each type of capture agent is immobilized to particles with the same ID, and the signals emitted from the detection molecules on the surface of the particles with a particular ID reflect the amount of the corresponding target.

Existing systems for suspension array multiplex assays are limited in resolution. Some multiplex systems use digital barcodes printed on flat microbeads using standard semiconductor fabrication techniques. However, the number of identifiers that can be generated by a particular number of digits is limited. Increasing the number of unique identifiers requires increasing the number of barcode digits, thus requiring more space for printing on an already tiny microbead. Another type of multiplex system uses color-coding, such as fluorescent beads encoded with a unique fluorescent dye. However, the number of unique identifiers available for such fluorescent systems is limited due to overlapping excitation/emission spectra, and identification errors may arise from, e.g., batch-to-batch variation in fluorescent dyes.

Therefore, a need exists for an analog-encoded multiplex assay system, e.g., one not constrained by limitations such as digital barcode size or fluorophore resolution. Such a system allows nearly unlimited unique identifiers and minimizes recognition error due to the use of analog codes (e.g., from overlapping spectra or batch-to-batch fluorophore variation). Moreover, a need exists for the microcarriers of such a system to be easily manipulated and/or separated, e.g., from a biological sample or other liquid.

All publications, patents, and patent applications cited herein are hereby incorporated by reference in their entirety for all purposes.

### BRIEF SUMMARY

To meet this need, provided herein, inter alia, are microcarriers, encoded with an analog code, that comprise a capture agent for capturing an analyte. The analog code is made from a two-dimensional shape of a substantially non-transparent layer, and the microcarrier also comprises a substantially transparent magnetic polymer layer. These features allow the microcarriers to be separated from a solution (e.g., a biological sample or assay solution) via magnetic attraction quickly and efficiently and to be fabricated using convenient microfabrication techniques. Moreover, the use of superparamagnetic materials such as certain iron oxides eliminates residual magnetism that can complicate an assay procedure. These microcarriers may be used, for example, in multiplexed assays in which each microcarrier comprises a capture agent for capturing a specific analyte and an analog code for identification. Methods of making and using such micocarriers, as well as kits related thereto, are further provided.

Accordingly, in one aspect, provided herein is an encoded microcarrier, comprising: (a) a substantially transparent magnetic polymer layer having a first surface and a second surface, the first and the second surfaces being parallel to each other, wherein the substantially transparent magnetic polymer comprises a mixture of a substantially transparent polymer and a plurality of magnetic nanoparticles, and wherein the magnetic nanoparticles comprise iron(II,III) oxide or iron(III) oxide; (b) a substantially non-transparent layer, wherein the substantially non-transparent layer is affixed to the first surface of the substantially transparent magnetic polymer layer, and wherein an outline of the substantially non-transparent layer constitutes a two-dimensional shape representing an analog code; and (c) a capture agent for capturing an analyte, wherein the capture agent is coupled to at least one of the first surface and the second surface of the substantially transparent magnetic polymer layer.

In some embodiments, the magnetic nanoparticles are superparamagnetic. In some embodiments, the magnetic nanoparticles are less than about 30 nm in diameter and greater than or equal to about 3 nm in diameter. In some embodiments, the plurality of magnetic nanoparticles comprises less than about 10% (by weight) and greater than about 0.1% (by weight) of the mixture of the substantially transparent polymer and the plurality of magnetic nanoparticles. In some embodiments, the substantially transparent magnetic polymer layer is between about 0.1 μm and about 50 μm in thickness. In some embodiments, the substantially transparent polymer is an epoxy-based polymer. In some embodiments, the epoxy-based polymer is SU-8. In some embodiments, the substantially non-transparent layer comprises a substantially non-transparent polymer. In some embodiments, the substantially non-transparent layer comprises a black matrix resist. In some embodiments, the substantially non-transparent polymer exhibits an absorbance of greater than about 1.8 (OD) at a wavelength between about 230 nm and about 660 nm. In some embodiments, the substantially non-transparent layer comprises a metal that lacks residual magnetism. In some embodiments, the substantially non-transparent layer comprises titanium or chromium. In some embodiments, the substantially non-transparent layer is between about 0.05 μm and about 2 μm in thickness. In some embodiments, the analog code comprises one or more overlapping or partially overlapping arc elements forming a continuous or discontinuous ring. In some embodiments, the microcarrier further comprises an orientation indicator for orienting the analog code of the substantially non-transparent layer. In some embodiments, the orientation indicator comprises an asymmetry of the substantially non-transparent layer. In some embodiments, the microcarrier is a substantially circular disc. In some embodiments, the microcarrier is between about 5 μm and about 200 μm in diameter. In some embodiments, the microcarrier is about 40 μm in diameter. In some embodiments, the microcarrier is less than about 50 μm in thickness. In some embodiments, the microcarrier is between about 2 μm and about 10 μm in thickness. In some embodiments, the microcarrier is about 5 μm in thickness.

In some embodiments, the analyte is selected from the group consisting of a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment. In some embodiments, the capture agent for capturing the analyte is selected from the group consisting of a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment.

In another aspect, provided herein are methods of making an encoded microcarrier, comprising: (a) depositing a substantially non-transparent layer having a first surface and a second surface, the first and the second surfaces being parallel to each other; (b) patterning the deposited substantially non-transparent layer into a two-dimensional shape representing an analog code; (c) depositing a substantially transparent magnetic polymer layer onto the first surface of the deposited substantially non-transparent layer, wherein the substantially transparent magnetic polymer layer comprises a mixture of a substantially transparent polymer and a plurality of magnetic nanoparticles, and wherein the magnetic nanoparticles comprise iron(II,III) oxide or iron(III) oxide; and (d) patterning the deposited substantially transparent magnetic polymer layer into a microcarrier shape. In some embodiments, (a) comprises depositing the substantially non-transparent layer onto a sacrificial layer. In some embodiments, the substantially non-transparent layer is deposited and patterned by lithography. In some embodiments, the deposited substantially non-transparent layer is deposited and patterned by lift-off process. In some embodiments, the method further comprises, prior to (a), depositing a second polymer layer onto the sacrificial layer; prior to (a), applying a mask to the deposited second polymer layer, thereby generating a masked portion of the deposited second polymer layer and a non-masked portion of the deposited second polymer layer; prior to (a), applying light to the masked, deposited second polymer layer, wherein the light is of a wavelength sufficient to remove the non-masked portion of the deposited second polymer layer; wherein (a) comprises depositing the substantially non-transparent layer onto the sacrificial layer and the masked portion of the deposited second polymer layer; and wherein (b) comprises removing the deposited second polymer layer, thereby patterning the deposited substantially non-transparent layer by removing the substantially non-transparent layer deposited onto the masked portion of the deposited second polymer layer. In some embodiments, the second polymer layer is deposited by spin coating. In some embodiments, the method further comprises, prior to (a)-(d), depositing the sacrificial layer onto a substrate carrier. In some embodiments, the method further comprises, after (a)-(d), etching out the sacrificial layer. In some embodiments, the method further comprises prior to (c), mixing monomer for the substantially transparent polymer, the plurality of magnetic nanoparticles, a solvent, and a dispersing agent to form a mixture of the monomer for the substantially transparent polymer, the plurality of magnetic nanoparticles, the solvent, and the dispersing agent.

In some embodiments, the plurality of magnetic nanoparticles, the solvent, and the dispersing agent are mixed to form a mixture of the plurality of magnetic nanoparticles, the solvent, and the dispersing agent, prior to mixing with the monomer for the substantially transparent polymer. In some embodiments, the dispersing agent comprises 10% of the mixture of the plurality of magnetic nanoparticles, the solvent, and the dispersing agent. In some embodiments, the plurality of magnetic nanoparticles comprises 2.5% of the mixture of the monomer for the substantially transparent polymer, the plurality of magnetic nanoparticles, the solvent, and the dispersing agent. In some embodiments, the solvent comprises cyclopentanone. In some embodiments, the dispersing agent comprises a phosphoric acid polymer. In some embodiments, the phosphoric acid polymer comprises carboxyl-PEG-phosphoric acid. In some embodiments, the substantially transparent magnetic polymer layer is deposited by spin coating or spray coating. In some embodiments, the deposited substantially transparent magnetic polymer layer is patterned by photolithography, lift-off, or sputtering.

In some embodiments, the method further comprises: coupling a capture agent for capturing an analyte to at least one of the first surface and the second surface of the substantially transparent magnetic polymer layer. In some embodiments, coupling the capture agent comprises: reacting the substantially transparent polymer of the substantially transparent magnetic polymer layer with a photoacid generator and light to generate a cross-linked polymer, wherein the light is of a wavelength that activates the photoacid generator; reacting the epoxide of the cross-linked polymer with a compound comprising an amine and a carboxyl, wherein the amine of the compound reacts with the epoxide to form a compound-coupled, cross-linked polymer; and reacting the carboxyl of the compound-coupled, cross-linked polymer with the capture agent to couple the capture agent to the substantially transparent magnetic polymer layer.

In some embodiments, the magnetic nanoparticles are superparamagnetic. In some embodiments, the magnetic nanoparticles are less than about 30 nm in diameter and greater than or equal to about 3 nm in diameter. In some embodiments, the plurality of magnetic nanoparticles comprises less than about 10% (by weight) and greater than about 0.1% (by weight) of the mixture of the substantially transparent polymer and the plurality of magnetic nanoparticles. In some embodiments, the substantially transparent magnetic polymer layer is between about 0.1 μm and about 50 μm in thickness. In some embodiments, the substantially transparent polymer of the substantially transparent magnetic polymer layer is an epoxy-based polymer. In some embodiments, the epoxy-based polymer is SU-8. In some embodiments, the substantially non-transparent layer comprises a substantially non-transparent polymer. In some embodiments, the substantially non-transparent layer comprises a black matrix resist. In some embodiments, the substantially non-transparent polymer exhibits an absorbance of greater than about 1.8 (OD) at a wavelength between about 230 nm and about 660 nm. In some embodiments, the substantially non-transparent layer comprises a metal that lacks residual magnetism. In some embodiments, the substantially non-transparent layer comprises titanium or chromium. In some embodiments, the substantially non-transparent layer is between about 0.05 μm and about 2 μm in thickness. In some embodiments, the analog code comprises one or more overlapping or partially overlapping arc elements forming a continuous or discontinuous ring. In some embodiments, the deposited substantially non-transparent layer is patterned so as to provide an asymmetry. In some embodiments, the microcarrier is patterned in (b) into a substantially circular disc. In some embodiments, the microcarrier is between about 5 μm and about 200 μm in diameter. In some embodiments, the microcarrier is about 40 μm in diameter. In some embodiments, the microcarrier is less than about 50 μm in thickness. In some embodiments, the microcarrier is between about 2 μm and about 10 μm in thickness.

In some embodiments, the analyte is selected from the group consisting of a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment.

In some embodiments, the capture agent for capturing the analyte is selected from the group consisting of a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment.

In one aspect, disclosed herein are encoded micocarriers produced by any of the methods provided herein.

In another aspect, disclosed herein are methods for detecting multiple analytes in a solution, comprising: contacting a solution comprising a first analyte and a second analyte with a plurality of microcarriers, wherein the plurality of microcarriers comprises at least: (i) a first microcarrier according to any of the embodiments described herein that specifically captures the first analyte, wherein the first microcarrier is encoded with a first analog code; and (ii) a second microcarrier according to any of the embodiments described herein that specifically captures the second analyte, wherein the second microcarrier is encoded with a second analog code, and wherein the second analog code is different from the first analog code; (b) decoding the first analog code and the second analog code using analog shape recognition to identify the first microcarrier and the second microcarrier; and (c) detecting an amount of the first analyte bound to the first microcarrier and an amount of the second analyte bound to the second microcarrier. In some embodiments, (b) occurs before (c). In some embodiments, (c) occurs before (b). In some embodiments, (b) and (c) occur simultaneously. In some embodiments, decoding the first analog code and the second analog code comprises: (i) illuminating the first and second microcarriers by passing light through the substantially transparent magnetic polymer layers of the first and second microcarriers and/or the surrounding solution, wherein the light fails to pass through the substantially non-transparent layers of the first and second microcarriers to generate a first analog-coded light pattern corresponding to the first microcarrier and a second analog-coded light pattern corresponding to the second microcarrier; (ii) imaging the first analog-coded light pattern to generate a first analog-coded image and imaging the second analog-coded light pattern to generate a second analog-coded image; and (iii) using analog shape recognition to match the first analog-coded image with the first analog code and to match the second analog-coded image with the second analog code.

In some embodiments, detecting the amount of the first analyte bound to the first microcarrier and the amount of the second analyte bound to the second microcarrier comprises: (i) after (a), incubating the first and the second microcarriers with a detection agent, wherein the detection agent binds the first analyte captured by the first microcarrier and the second analyte captured by the second microcarrier; and (ii) measuring the amount of detection agent bound to the first and the second microcarriers. In some embodiments, the detection agent is a fluorescent detection agent, and wherein the amount of detection agent bound to the first and the second microcarriers is measured by fluorescence microscopy. In some embodiments, the detection agent is a luminescent detection agent, and wherein the amount of detection agent bound to the first and the second microcarriers is measured by luminescence microscopy. In some embodiments, the solution comprises a biological sample. In some embodiments, the biological sample is selected from the group consisting of blood, urine, sputum, bile, cerebrospinal fluid, interstitial fluid of skin or adipose tissue, saliva, tears, bronchial-alveolar lavage, oropharyngeal secretions, intestinal fluids, cervico-vaginal or uterine secretions, and seminal fluid.

In one aspect, described herein are kits for conducting a multiplex assay comprising a plurality of microcarriers, wherein the plurality of microcarriers comprises at least: (a) a first microcarrier according to any of the embodiments described herein that specifically captures a first analyte, wherein the first microcarrier is encoded with a first analog code; and (b) a second microcarrier according to any of the embodiments described herein that specifically captures a second analyte, wherein the second microcarrier is encoded with a second analog code, and wherein the second analog code is different from the first analog code.

In some embodiments, the kit further comprises a detection agent for detecting an amount of the first analyte bound to the first microcarrier and an amount of the second analyte bound to the second microcarrier. In some embodiments, the kit further comprises instructions for using the kit to detect the first analyte and the second analyte.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIGS. 1A & 1B show two views of an exemplary microcarrier.

FIGS. 1C & 1D show an exemplary assay for analyte detection using an exemplary microcarrier.

FIG. 2A shows three examples of microcarriers, each having a unique analog code.

FIG. 2B shows examples of microcarriers with a unique analog code, in accordance with some embodiments.

FIG. 2C shows an example of a microcarrier with a unique analog code, in accordance with some embodiments.

FIG. 3 shows a method for producing an exemplary microcarrier.

FIG. 4 shows a method for producing an exemplary microcarrier. Overhead (left) and cross-sectional (right) views are shown.

### DETAILED DESCRIPTION

In one aspect, provided herein are encoded microcarriers for analyte detection in multiplex assays. In some embodiments, the microcarriers comprise (a) a substantially transparent magnetic polymer layer having a first surface and a second surface, the first and the second surfaces being parallel to each other, wherein the substantially transparent magnetic polymer comprises a mixture of a substantially transparent polymer and a plurality of magnetic nanoparticles, and wherein the magnetic nanoparticles comprise iron(II,III) oxide or iron(III) oxide; (b) a substantially non-transparent layer, wherein the substantially non-transparent layer is affixed to the first surface of the substantially transparent magnetic polymer layer, and wherein an outline of the substantially non-transparent layer constitutes a two-dimensional shape representing an analog code; and (c) a capture agent for capturing an analyte, wherein the capture agent is coupled to at least one of the first surface and the second surface of the substantially transparent magnetic polymer layer.

In another aspect, provided herein are methods of making encoded microcarriers, comprising: (a) depositing a substantially non-transparent layer having a first surface and a second surface, the first and the second surfaces being parallel to each other; (b) patterning the deposited substantially non-transparent layer into a two-dimensional shape representing an analog code; (c) depositing a substantially transparent magnetic polymer layer onto the first surface of the deposited substantially non-transparent layer, wherein the substantially transparent magnetic polymer layer comprises a mixture of a substantially transparent polymer and a plurality of magnetic nanoparticles, and wherein the magnetic nanoparticles comprise iron(II,III) oxide or iron(III) oxide; and (d) patterning the deposited substantially transparent magnetic polymer layer into a microcarrier shape.

In some embodiments, further provided herein are encoded microcarriers produced by the methods disclosed herein.

In still another aspect, provided herein are methods for detecting multiple analytes in a solution by (a) contacting a solution comprising a first analyte and a second analyte with a plurality of microcarriers, where the plurality of microcarriers comprises at least: (i) a first microcarrier of the present disclosure that specifically captures the first analyte, where the first microcarrier is encoded with a first analog code; and (ii) a second microcarrier of the present disclosure that specifically captures the second analyte, where the second microcarrier is encoded with a second analog code, and where the second analog code is different from the first analog code; (b) decoding the first analog code and the second analog code using analog shape recognition to identify the first microcarrier and the second microcarrier; and (c) detecting an amount of the first analyte bound to the first microcarrier and an amount of the second analyte bound to the second microcarrier.

In yet another aspect, provided herein are kits or articles of manufacture for conducting a multiplex assay comprising a plurality of microcarriers. The plurality of microcarriers comprises at least (a) a first microcarrier of the present disclosure that specifically captures a first analyte, where the first microcarrier is encoded with a first analog code; and (b) a second microcarrier of the present disclosure that specifically captures a second analyte, where the second microcarrier is encoded with a second analog code, and where the second analog code is different from the first analog code.

### I. General Techniques

The practice of the techniques described herein will employ, unless otherwise indicated, conventional techniques in polymer technology, microfabrication, micro-electro-mechanical systems (MEMS) fabrication, photolithography, microfluidics, organic chemistry, biochemistry, oligonucleotide synthesis and modification, bioconjugate chemistry, nucleic acid hybridization, molecular biology, microbiology, genetics, recombinant DNA, and related fields as are within the skill of the art. The techniques are described in the references cited herein and are fully explained in the literature.

For molecular biology and recombinant DNA techniques, see, for example, (Maniatis, T. et al. (1982), Molecular Cloning: A Laboratory Manual, Cold Spring Harbor; Ausubel, F. M. (1987), Current Protocols in Molecular Biology, Greene Pub. Associates and Wiley-Interscience; Ausubel, F. M. (1989), Short Protocols in Molecular Biology: A Compendium of Methods from Current Protocols in Molecular Biology, Greene Pub. Associates and Wiley-Interscience; Sambrook, J. et al. (1989), Molecular Cloning: A Laboratory Manual, Cold Spring Harbor; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates.

For DNA synthesis techniques and nucleic acids chemistry, see for example, Gait, M. J. (1990), Oligonucleotide Synthesis: A Practical Approach, IRL Press; Eckstein, F. (1991), Oligonucleotides and Analogues: A Practical Approach, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press).

For microfabrication, see for example, (Campbell, S. A. (1996), The Science and Engineering of Microelectronic Fabrication, Oxford University Press; Zaut, P. V. (1996), Microarray Fabrication: a Practical Guide to Semiconductor Processing, Semiconductor Services; Madou, M. J. (1997), Fundamentals of Microfabrication, CRC Press; Rai-Choudhury, P. (1997). Handbook of Microlithography, Micromachining, & Microfabrication: Microlithography).

### II. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

The term “microcarrier” as used herein may refer to a physical substrate onto which a capture agent may be coupled. A microcarrier of the present disclosure may take any suitable geometric form or shape. In some embodiments, the microcarrier may be disc-shaped. Typically the form or shape of a microcarrier will comprise at least one dimension on the order of 10⁻⁴ to 10⁻⁷ m (hence the prefix “micro”).

The term “polymer” as used herein may refer to any macromolecular structure comprising repeated monomers. A polymer may be natural (e.g., found in nature) or synthetic (e.g., man-made, such as a polymer composed of non-natural monomer(s) and/or polymerized in a configuration or combination not found in nature).

The terms “substantially transparent” and “substantially non-transparent” as used herein may refer to the ability of light (e.g., of a particular wavelength, such as infrared, visible, UV, and so forth) to pass through a substrate, such as a polymer layer. A substantially transparent polymer may refer to one that is transparent, translucent, and/or pervious to light, whereas a substantially non-transparent polymer may refer to one that reflects and/or absorbs light. It is to be appreciated that whether a material is substantially transparent or substantially non-transparent may depend upon the wavelength and/or intensity of light illuminating the material, as well as the means detecting the light traveling through the material (or a decrease or absence thereof). In some embodiments, a substantially non-transparent material causes a perceptible decrease in transmitted light as compared to the surrounding material or image field, e.g., as imaged by light microscopy (e.g., bright field, dark field, phase contrast, differential interference contrast (DIC), Nomarski interference contrast (NIC), Nomarski, Hoffman modulation contrast (HMC), or fluorescence microscopy). In some embodiments, a substantially transparent material allows a perceptible amount of transmitted light to pass through the material, e.g., as imaged by light microscopy (e.g., bright field, dark field, phase contrast, differential interference contrast (DIC), Nomarski interference contrast (NIC), Nomarski, Hoffman modulation contrast (HMC), or fluorescence microscopy).

The term “analog code” as used herein may refer to any code in which the encoded information is represented in a non-quantized and/or non-discrete manner, e.g., as opposed to a digital code. For example, a digital code is sampled at discrete positions for a limited set of values (e.g., 0/1 type values), whereas an analog code may be sampled at a greater range of positions (or as a continuous whole) and/or may contain a wider set of values (e.g., shapes). In some embodiments, an analog code may be read or decoded using one or more analog shape recognition techniques.

The term “capture agent” as used herein is a broad term and is used in its ordinary sense to refer to any compound or substance capable of specifically recognizing an analyte of interest. In some embodiments, specific recognition may refer to specific binding. Non-limiting examples of capture agents comprise, for example, a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment.

“Analyte,” as used herein, is a broad term and is used in its ordinary sense as a substance the presence, absence, or quantity of which is to be determined, comprising, without limitation, to refer to a substance or chemical constituent in a sample such as a biological sample or cell or population of cells that can be analyzed. An analyte can be a substance for which a naturally occurring binding member exists, or for which a binding member can be prepared. Non-limiting examples of analytes comprise, for example, antibodies, antibody fragments, antigens, polynucleotides (such as a DNA molecule, DNA-analog-molecule, RNA-molecule, or RNA-analog-molecule), polypeptides, proteins, enzymes, lipids, phospholipids, carbohydrate moieties, polysaccharides, small molecules, organelles, hormones, cytokines, growth factors, steroids, vitamins, toxins, drugs, and metabolites of the above substances, as well as cells, bacteria, viruses, fungi, algae, fungal spores and the like.

The term “antibody” is used in the broadest sense and comprises monoclonal antibodies (comprising full length antibodies which have an immunoglobulin Fc region), polyclonal antibodies, multispecific antibodies (e.g., bispecific antibodies, diabodies, and single-chain molecules), as well as antibody fragments (e.g., Fab, F(ab′)₂, and Fv).

As used herein, “sample” refers to a composition containing a material, such as a molecule, to be detected. In one embodiment, the sample is a “biological sample” (i.e., any material obtained from a living source (e.g. human, animal, plant, bacteria, fungi, protist, virus)). The biological sample can be in any form, comprising solid materials (e.g. tissue, cell pellets and biopsies) and biological fluids (e.g. urine, blood, saliva, lymph, tears, sweat, prostatic fluid, seminal fluid, semen, bile, mucus, amniotic fluid and mouth wash (containing buccal cells)). Solid materials typically are mixed with a fluid. Sample can also refer to an environmental sample such as water, air, soil, or any other environmental source.

As used in this specification and the appended claims, the singular forms “a”, “an” and “the” comprise plural referents unless the content clearly dictates otherwise. Thus, for example, reference to “a molecule” optionally comprises a combination of two or more such molecules, and the like.

The term “about” as used herein refers to the usual error range for the respective value readily known to the skilled person in this technical field. Reference to “about” a value or parameter herein comprises (and describes) embodiments that are directed to that value or parameter per se.

It is understood that aspects and embodiments of the invention described herein comprise “comprising,” “consisting,” and “consisting essentially of” aspects and embodiments.

### III. Encoded Microcarriers

Provided herein are encoded microcarriers suitable for analyte detection, e.g., multiplex analyte detection. Multiple configurations for encoded microcarriers are contemplated, described, and exemplified herein.

In some aspects, provided herein are encoded microcarriers that comprise: a substantially transparent magnetic polymer layer comprising a mixture of a substantially transparent polymer and a plurality of magnetic nanoparticles and a substantially non-transparent layer whose outline constitutes a two-dimensional shape representing an analog code. In some embodiments, the microcarriers further comprise a capture agent for capturing an analyte that is coupled to the substantially transparent magnetic polymer layer (e.g., on one or both surfaces). In some embodiments, the magnetic nanoparticles comprise iron(II,III) oxide or iron(III) oxide. Configurations, parameters, and optional features of the encoded microcarriers of the present disclosure are provided in the non-limiting descriptions infra.

In some embodiments, a substantially transparent magnetic polymer layer of the present disclosure comprises a mixture of a substantially transparent polymer and a plurality of magnetic nanoparticles. In some embodiments, the substantially transparent magnetic polymer layer comprises a suspension of magnetic nanoparticles in a substantially transparent polymer, e.g., an epoxy-based polymer. For example, the magnetic nanoparticles can be made into a stable dispersion (e.g., with the use of a solvent comprising but not limited to cyclopentanone or g-Butyrolactone, and/or a dispersing agent comprising but not limited to phosphoric acid polymers) and mixed with a dissolved polymer or monomeric form of the polymer. Incorporating magnetic properties into the substantially transparent polymer layer has advantages over using a dedicated magnetic layer (such as, e.g., a magnetic ring) by allowing for a reduced overall size (e.g., area) of the microcarriers, thereby increasing the number of microcarriers that can be produced from a single wafer and reducing the per-unit cost. In addition, a magnetic substantially transparent polymer layer does not need to be sandwiched between polymer layers to insulate it from the environment, as other dedicated magnetic layers (e.g., nickel layers) may require. This simplified, two-layer model reduces manufacturing costs and increases manufacturing consistency without sacrificing functionality. Exemplary descriptions and techniques related to magnetic nanoparticle suspensions and mixing the same with epoxy-based polymers may be found, e.g., in Suter, Marcel. Photopatternable superparamagnetic nanocomposite for the fabrication of microstructures. Diss. ETH Zurich, 2011; and Suter, M. (2011) Sensors and Actuators B: Chemical 156:433-43.

In some embodiments, the magnetic nanoparticles are superparamagnetic nanoparticles. Without wishing to be bound to theory, it is thought that such a material is advantageous for microcarrier fabrication because the resulting microcarriers display no residual magnetism and/or low magnetic attraction with each other (as opposed to magnetic materials with some residual magnetism after removal of the magnetic field, such as nickel), thus preventing them from interacting detrimentally with each other in solution. It is also thought that such materials are uniquely suited for analog encoding (e.g., as described herein) because the materials (e.g., an SU-8/iron(II,III) oxide or iron(III) oxide nanoparticle mixture or suspension, which can replace the use of nickel or rare earth metals) allow for very thin magnetic layers with enhanced image recognizability (e.g., when imaged as described herein). These microcarriers are also thought to be simpler for fabrication. In certain embodiments, the magnetic nanoparticles comprise iron(III) oxide (Fe₂O₃; also known as ferric oxide or maghemite) and/or iron(II,III) oxide (Fe₃O₄; also known as magnetite). In certain embodiments, the epoxy-based polymer is SU-8.

In some embodiments, magnetic nanoparticles of the present disclosure are less than about 30 nm in diameter. In some embodiments, magnetic nanoparticles of the present disclosure are greater than or equal to about 3 nm in diameter. In some embodiments, magnetic nanoparticles of the present disclosure can be any size (e.g., diameter) less than about any of the following sizes (in nm): 30, 25, 20, 15, 10, or 5. In some embodiments, magnetic nanoparticles of the present disclosure can be any size (e.g., diameter) greater than or equal to about any of the following sizes (in nm): 3, 5, 10, 15, 20, or 25. That is, the magnetic nanoparticles can be of any size (e.g., diameter) having an upper limit of 30, 25, 20, 15, 10, or 5 nm and an independently selected lower limit of 3, 5, 10, 15, 20, or 25 nm, wherein the lower limit is less than the upper limit.

In some embodiments, a substantially transparent magnetic polymer layer comprises a mixture of a substantially transparent polymer and a plurality of magnetic nanoparticles, wherein the plurality of magnetic nanoparticles comprises less than about 10% (by weight) and/or greater than about 0.1% (by weight) of the mixture. In some embodiments, the plurality of magnetic nanoparticles comprises around 2.5% (by weight) of the mixture. In some embodiments, a substantially transparent magnetic polymer layer of the present disclosure comprises a mixture comprising a plurality of magnetic nanoparticles at a concentration less than about any of the following concentrations (% by weight): 10, 9, 8, 7, 6, 5, 4, 3, 2, 1, 0.9, 0.8, 0.7, 0.6, 0.5, 0.4, or 0.3. In some embodiments, a substantially transparent magnetic polymer layer of the present disclosure comprises a mixture comprising a plurality of magnetic nanoparticles at a concentration greater than about any of the following concentrations (% by weight): 0.1, 0.2, 0.3, 0.4, 0.5, 0.6, 0.7, 0.8, 0.9, 1, 2, 3, 4, 5, 6, 7, or 8. That is, a substantially transparent magnetic polymer layer of the present disclosure can comprise a mixture comprising a plurality of magnetic nanoparticles at a concentration having an upper limit of 10, 9, 8, 7, 6, 5, 4, 3, 2, 1, 0.9, 0.8, 0.7, 0.6, 0.5, 0.4, or 0.3% and an independently selected lower limit of 0.1, 0.2, 0.3, 0.4, 0.5, 0.6, 0.7, 0.8, 0.9, 1, 2, 3, 4, 5, 6, 7, or 8%, wherein the lower limit is less than the upper limit.

In some embodiments, a substantially transparent polymer of the present disclosure comprises an epoxy-based polymer. Suitable epoxy-based polymers for fabrication of the compositions described herein comprise, but are not limited to, the EPON™ family of epoxy resins provided by Hexion Specialty Chemicals, Inc. (Columbus, OH) and any number of epoxy resins provided by The Dow Chemical Company (Midland, MI). Many examples of suitable polymers are commonly known in the art, comprising without limitation SU-8, EPON 1002F, EPON 165/154, and a poly(methyl methacrylate)/poly(acrylic acid) block copolymer (PMMA-co-PAA). For additional polymers, see, for example, Warad, IC Packaging: Package Construction Analysis in Ultra Small IC Packaging, LAP LAMBERT Academic Publishing (2010); The Electronic Packaging Handbook, CRC Press (Blackwell, ed.), (2000); and Pecht et al., Electronic Packaging Materials and Their Properties, CCR Press, 1^{st} ed., (1998). These types of materials have the advantage of not swelling in aqueous environments which ensures that uniform microcarrier size and shape are maintained within the population of microcarriers. In some embodiments, the substantially transparent polymer is a photoresist polymer. In some embodiments, the epoxy-based polymer is an epoxy-based, negative-tone, near-UV photoresist. In some embodiments, the epoxy-based polymer is SU-8.

In some embodiments, a microcarrier of the present disclosure comprises a substantially transparent magnetic polymer layer that is greater than or equal to about 0.1 μm in thickness. In some embodiments, a microcarrier of the present disclosure comprises a substantially transparent magnetic polymer layer that is less than or equal to about 50 μm in thickness. In some embodiments, a microcarrier of the present disclosure comprises a substantially transparent magnetic polymer layer that is between about 0.1 μm and about 50 μm in thickness. In some embodiments, a microcarrier of the present disclosure comprises a substantially transparent magnetic polymer layer with a thickness that is less than or equal to about any of the following thicknesses (in μm): 50, 45, 40, 35, 30, 25, 20, 15, 10, 9, 8, 7, 6, 5, 4, 3, 2, 1, 0.9, 0.8, 0.7, 0.6, 0.5, 0.4, or 0.3. In some embodiments, a microcarrier of the present disclosure comprises a substantially transparent magnetic polymer layer with a thickness that is greater than or equal to about any of the following thicknesses (in μm): 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, or 45. That is, a microcarrier of the present disclosure can comprise a substantially transparent magnetic polymer layer with a thickness having an upper limit of 50, 45, 40, 35, 30, 25, 20, 15, 10, 9, 8, 7, 6, 5, 4, 3, 2, 1, 0.9, 0.8, 0.7, 0.6, 0.5, 0.4, or 0.3 μm and an independently selected lower limit of 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, or 45 μm, wherein the lower limit is less than the upper limit.

In some embodiments, a microcarrier of the present disclosure comprises a substantially non-transparent layer. For example, the substantially non-transparent layer can be made of a substantially non-transparent polymer, or a metal.

In some embodiments, the substantially non-transparent layer comprises a polymer described herein (e.g., SU-8) mixed with one or more non-transparent or colored dye(s). In other embodiments, the substantially non-transparent layer comprises a black matrix resist. Any black matrix resist known in the art may be used; see, e.g., U.S. Pat. No. 8,610,848 for exemplary black matrix resists and methods related thereto. In some embodiments, the black matrix resist may be a photoresist colored with a black pigment, e.g., as patterned on the color filter of an LCD as part of a black matrix. Black matrix resists may comprise without limitation those sold by Toppan Printing Co. (Tokyo), Tokyo OHKA Kogyo (Kawasaki), and Daxin Materials Corp. (Taichung City, Taiwan).

In some embodiments, the substantially non-transparent layer comprises a substantially non-transparent polymer that exhibits an absorbance of greater than about 1.8 (OD) at a wavelength between about 230 nm and about 660 nm. For example, the polymer can exhibit an absorbance of greater than about 1.8 (OD) at one or more wavelengths selected from the group consisting of 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, and 660 nm.

In some embodiments, the substantially non-transparent layer comprises a metal. In some embodiments, the metal lacks residual magnetism. In some embodiments, the substantially non-transparent layer comprises nickel, titanium, copper, and/or chromium.

In some embodiments, a microcarrier of the present disclosure comprises a substantially non-transparent layer that is greater than or equal to about 0.05 μm in thickness. In some embodiments, a microcarrier of the present disclosure comprises a substantially non-transparent layer that is less than or equal to about 2 μm in thickness. In some embodiments, a microcarrier of the present disclosure comprises a substantially non-transparent layer that is between about 0.05 μm and about 2 μm in thickness. In some embodiments, a microcarrier of the present disclosure comprises a substantially non-transparent layer with a thickness that is less than or equal to about any of the following thicknesses (in μm): 2, 1.9, 1.8, 1.7, 1.6, 1.5, 1.4, 1.3, 1.2, 1.1, 1.0, 0.9, 0.8, 0.7, 0.6, 0.5, 0.45, 0.4, 0.35, 0.3, 0.25, 0.2, 0.15, 0.125, 0.1, or 0.075. In some embodiments, a microcarrier of the present disclosure comprises a substantially non-transparent layer with a thickness that is greater than or equal to about any of the following thicknesses (in μm): 0.05, 0.075, 0.1, 0.125, 0.15, 0.2, 0.25, 0.3, 0.35, 0.4, 0.45, 0.5, 0.6, 0.7, 0.8, 0.9, 1.0, 1.1, 1.2, 1.3, 1.4, 1.5, 1.6, 1.7, or 1.8. That is, a microcarrier of the present disclosure can comprise a substantially non-transparent layer with a thickness having an upper limit of 2, 1.9, 1.8, 1.7, 1.6, 1.5, 1.4, 1.3, 1.2, 1.1, 1.0, 0.9, 0.8, 0.7, 0.6, 0.5, 0.45, 0.4, 0.35, 0.3, 0.25, 0.2, 0.15, 0.125, 0.1, or 0.075 μm and an independently selected lower limit of 0.05, 0.075, 0.1, 0.125, 0.15, 0.2, 0.25, 0.3, 0.35, 0.4, 0.45, 0.5, 0.6, 0.7, 0.8, 0.9, 1.0, 1.1, 1.2, 1.3, 1.4, 1.5, 1.6, 1.7, or 1.8 μm, wherein the lower limit is less than the upper limit.

In some embodiments, a microcarrier of the present disclosure may be encoded with a substantially non-transparent layer that constitutes a two-dimensional shape. For example, as described above, the two-dimensional shape may constitute the shape of a substantially non-transparent layer that contrasts with a substantially transparent layer of the microcarrier, or it may constitute the shape of the microcarrier itself (e.g., the perimeter). That is to say, the code is the shape of the substantially non-transparent layer itself (e.g., rather than a code generated by a fluorescent or other visible moiety on the surface of a layer of the microcarrier). Any two-dimensional shape that can encompass a plurality of resolvable and distinctive varieties may be used. In some embodiments, the two-dimensional shape comprises one or more of linear, circular, elliptical, rectangular, quadrilateral, or higher polygonal aspects, elements, and/or shapes.

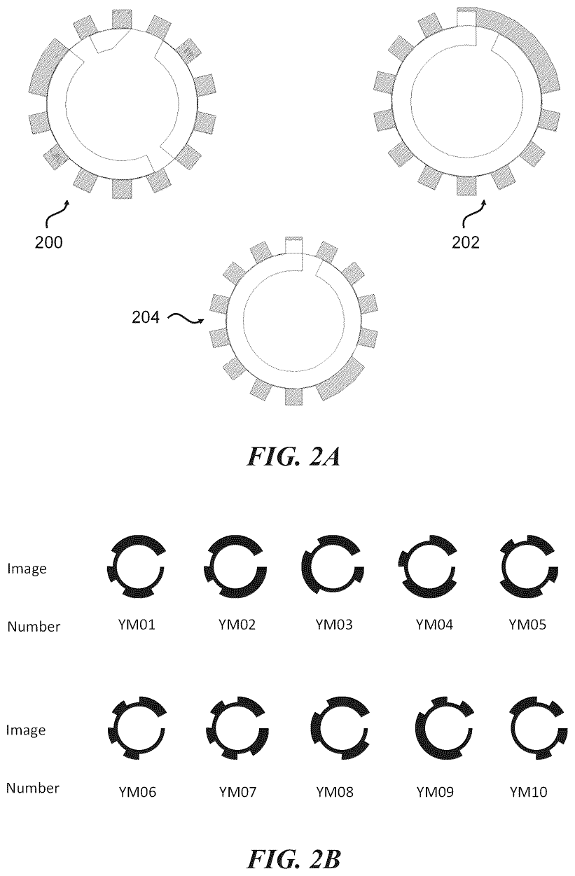

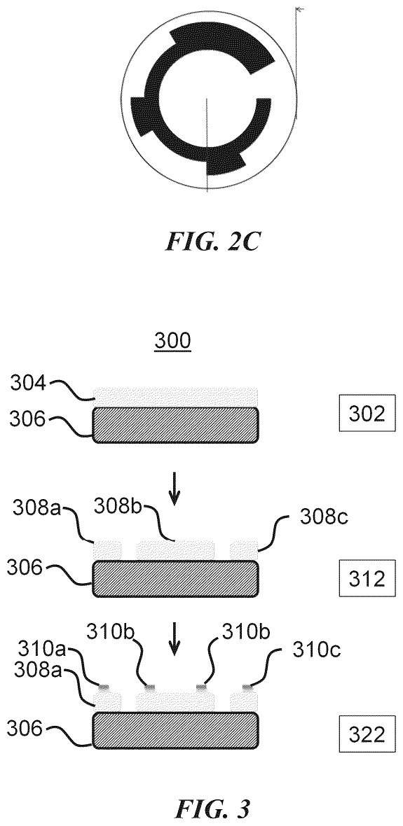

In some embodiments, the two-dimensional shape of the substantially non-transparent polymer layer comprises one or more rings enclosing the center portion of the substantially transparent polymer layer. In some embodiments, at least one of the one or more rings comprises a discontinuity. Exemplary and non-limiting two-dimensional shapes formed using one or more rings (e.g., two rings) having varying numbers and configurations of discontinuities are illustrated in FIG. 2B.

In some embodiments, the analog code comprises one or more overlapping or partially overlapping arc elements forming a continuous or discontinuous ring (e.g., surrounding a center portion of the microcarrier). The two-dimensional shape is decoded by imaging the microcarrier (e.g., by light microscopy), such that an image of the code is formed by the pattern generated by light passed through the substantially transparent magnetic polymer layer and light blocked from passing through the substantially non-transparent layer. A non-limiting example of a two-dimensional shape made of overlapping arc elements that form a discontinuous ring is illustrated in FIG. 2C.

In some embodiments, the two-dimensional shape of the substantially non-transparent polymer layer comprises a gear shape (see, e.g., FIG. 2A). A gear shape as used herein may refer to a plurality of shapes (e.g., gear teeth) arrayed on the perimeter of a substantially round, elliptical, or circular body, where at least two of the shapes of the plurality are spatially separated. In some embodiments, the gear shape comprises a plurality of gear teeth. In some embodiments, the analog code is represented by one or more aspects selected from the height of one or more gear teeth of the plurality, the width of one or more gear teeth of the plurality, the number of gear teeth in the plurality, and the arrangement of one or more gear teeth within the plurality. Advantageously, a gear shape encompasses multiple aspects, comprising the height of gear teeth, the width of gear teeth, the number of gear teeth, and the arrangement of gear teeth, that may be varied in order to generate a large diversity of potential unique two-dimensional shapes. It is to be appreciated, however, that since the gear shapes of the present disclosure are used for encoding and are not required to physically intermesh with another gear (e.g., as with mechanical gears that transmit torque), gear teeth of the present disclosure are not constrained by the need for identical or intermeshing shapes, either within one gear shape or between multiple gear shapes. As such, the variety of shapes that may be considered a gear tooth of the present disclosure is significantly greater than with a mechanical gear.

In some embodiments, the plurality of gear teeth comprises one or more gear teeth that are between about 1 μm and about 10 μm wide. In some embodiments, the plurality of gear teeth comprises one or more gear teeth that are about 1 μm wide, about 1.5 μm wide, about 2 μm wide, about 2.5 μm wide, about 3μm wide, about 3.5 μm wide, about 4 μm wide, about 4.5 μm wide, about 5 μm wide, about 5.5 μm wide, about 6 μm wide, about 6.5 μm wide, about 7 μm wide, about 7.5 μm wide, about 8 μm wide, about 8.5 μm wide, about 9 μm wide, about 9.5 μm wide, or about 10 μm wide. In some embodiments, the plurality of gear teeth comprises one or more gear teeth that are less than about any of the following widths (in μm): 10, 9.5, 9, 8.5, 8, 7.5, 7, 6.5, 6, 5.5, 5, 4.5, 4, 3.5, 3, 2.5, 2, or 1.5. In some embodiments, the plurality of gear teeth comprises one or more gear teeth that are greater than about any of the following widths (in μm): 1, 1.5, 2, 2.5, 3, 3.5, 4, 4.5, 5, 5.5, 6, 6.5, 7, 7.5, 8, 8.5, 9, or 9.5. That is, the plurality of gear teeth may comprise one or more gear teeth that can be any of a range of widths having an upper limit of 10, 9.5, 9, 8.5, 8, 7.5, 7, 6.5, 6, 5.5, 5, 4.5, 4, 3.5, 3, 2.5, 2, or 1.5 and an independently selected lower limit of 1, 1.5, 2, 2.5, 3, 3.5, 4, 4.5, 5, 5.5, 6, 6.5, 7, 7.5, 8, 8.5, 9, or 9.5, wherein the lower limit is less than the upper limit.

In some embodiments, the plurality of gear teeth comprises one or more gear teeth that are between about 1 μm and about 10 μm tall. In some embodiments, the plurality of gear teeth comprises one or more gear teeth that are about 1 μm tall, about 1.5 μm tall, about 2 μm tall, about 2.5 μm tall, about 3 μm tall, about 3.5 μm tall, about 4 μm tall, about 4.5 μm tall, about 5 μm tall, about 5.5 μm tall, about 6 μm tall, about 6.5 μm tall, about 7 μm tall, about 7.5 μm tall, about 8 μm tall, about 8.5 μm tall, about 9 μm tall, about 9.5 μm tall, or about 10 μm tall. In some embodiments, the plurality of gear teeth comprises one or more gear teeth that are less than about any of the following heights (in μm): 10, 9.5, 9, 8.5, 8, 7.5, 7, 6.5, 6, 5.5, 5, 4.5, 4, 3.5, 3, 2.5, 2, or 1.5. In some embodiments, the plurality of gear teeth comprises one or more gear teeth that are greater than about any of the following heights (in μm): 1, 1.5, 2, 2.5, 3, 3.5, 4, 4.5, 5, 5.5, 6, 6.5, 7, 7.5, 8, 8.5, 9, or 9.5. That is, the plurality of gear teeth may comprise one or more gear teeth that can be any of a range of heights having an upper limit of 10, 9.5, 9, 8.5, 8, 7.5, 7, 6.5, 6, 5.5, 5, 4.5, 4, 3.5, 3, 2.5, 2, or 1.5 and an independently selected lower limit of 1, 1.5, 2, 2.5, 3, 3.5, 4, 4.5, 5, 5.5, 6, 6.5, 7, 7.5, 8, 8.5, 9, or 9.5, wherein the lower limit is less than the upper limit. It is to be appreciated that a gear tooth may have different measurable heights, depending on the point of reference, if the adjacent perimeter segments from which the gear tooth extends are uneven.

In some embodiments, the plurality of gear teeth comprises one or more gear teeth that are spaced between about 1 μm and about 10 μm apart. In some embodiments, the plurality of gear teeth comprises one or more gear teeth that are spaced about 1 μm apart, about 1.5 μm apart, about 2 μm apart, about 2.5 μm apart, about 3 μm apart, about 3.5 μm apart, about 4 μm apart, about 4.5 μm apart, about 5 μm apart, about 5.5 μm apart, about 6 μm apart, about 6.5 μm apart, about 7 μm apart, about 7.5 μm apart, about 8 μm apart, about 8.5 μm apart, about 9 μm apart, about 9.5 μm apart, or about 10 μm apart. In some embodiments, the plurality of gear teeth comprises one or more gear teeth that are spaced less than about any of the following widths apart (in μm): 10, 9.5, 9, 8.5, 8, 7.5, 7, 6.5, 6, 5.5, 5, 4.5, 4, 3.5, 3, 2.5, 2, or 1.5. In some embodiments, the plurality of gear teeth comprises one or more gear teeth that are spaced greater than about any of the following widths apart (in μm): 1, 1.5, 2, 2.5, 3, 3.5, 4, 4.5, 5, 5.5, 6, 6.5, 7, 7.5, 8, 8.5, 9, or 9.5. That is, the plurality of gear teeth may comprise one or more gear teeth that can be spaced any of a range of widths apart having an upper limit of 10, 9.5, 9, 8.5, 8, 7.5, 7, 6.5, 6, 5.5, 5, 4.5, 4, 3.5, 3, 2.5, 2, or 1.5 and an independently selected lower limit of 1, 1.5, 2, 2.5, 3, 3.5, 4, 4.5, 5, 5.5, 6, 6.5, 7, 7.5, 8, 8.5, 9, or 9.5, wherein the lower limit is less than the upper limit.

In some embodiments, the microcarrier further comprises an orientation indicator for orienting the analog code of the substantially non-transparent layer. Any feature of the microcarrier that is visible and/or detectable by imaging (e.g., a form of microscopic or other imaging described herein) and/or by image recognition software may serve as an orientation indicator. An orientation indicator may serve as a point of reference, e.g., for an image recognition algorithm, to orient the image of an analog code in a uniform orientation (i.e., the shape of the substantially non-transparent layer). Advantageously, this simplifies image recognition, as the algorithm would only need to compare the image of a particular analog code against a library of analog codes in the same orientation, and not against a library comprising all analog codes in all possible orientations. In some embodiments, the orientation indicator comprises an asymmetry of the substantially non-transparent layer, such as a discontinuity of its outline or shape. For example, the orientation indicator may comprise a visible feature, such as an asymmetry, of the two-dimensional shape representing the analog code of the microcarrier (e.g., as illustrated in FIGS. 2A-2C).

In some embodiments, a microcarrier of the present disclosure is a substantially circular disc. As used herein, a substantially circular shape may refer to any shape having a roughly identical distance between all of the points of the shape's perimeter and the shape's geometric center. In some embodiments, a shape is considered to be substantially circular if the variation among any of the potential radii connecting the geometric center and a given point on the perimeter exhibit 10% or lesser variation in length. As used herein, a substantially circular disc may refer to any substantially circular shape wherein the thickness of the shape is significantly less than its diameter. For example, in some embodiments, the thickness of a substantially circular disc may be less than about 50%, less than about 40%, less than about 30%, less than about 20%, less than about 15%, less than about 10%, or less than about 5% of its diameter. In certain embodiments, the thickness of the substantially circular disc may about 20% of its diameter. It is to be appreciated that the microcarriers of the present disclosure whose outline is a gear shape may also be considered substantially circular discs; for example, the shape of the microcarrier excluding the one or more gear teeth may comprise a substantially circular disc.

In some embodiments, the microcarrier is less than about 200 μm in diameter. For example, in some embodiments, the diameter of the microcarrier is less than about 200 μm, less than about 180 μm, less than about 160 μm, less than about 140 μm, less than about 120 μm, less than about 100 μm, less than about 80 μm, less than about 60 μm, less than about 40 μm, or less than about 20 μm. In some embodiments, the diameter of the microcarrier is greater than about 5 μm, greater than about 10 μm, greater than about 20 μm, greater than about 30 μm, greater than about 40 μm, greater than about 50 μm, greater than about 60 μm, greater than about 70 μm, greater than about 80 μm, greater than about 90 μm, greater than about 100 μm, greater than about 120 μm, greater than about 140 μm, or greater than about 150 μm. In some embodiments, a microcarrier of the present disclosure can be any size (e.g., diameter) less than about any of the following sizes (in μm): 200, 180, 160, 140, 120, 100, 80, 60, 40, or 20. In some embodiments, magnetic nanoparticles of the present disclosure can be any size (e.g., diameter) greater than or equal to about any of the following sizes (in μm): 5, 10, 20, 30, 40, 50, 60, 70, 80, 90, 100, 120, 140, or 150. That is, the magnetic nanoparticles can be of any size (e.g., diameter) having an upper limit of 200, 180, 160, 140, 120, 100, 80, 60, 40, or 20 μm and an independently selected lower limit of 5, 10, 20, 30, 40, 50, 60, 70, 80, 90, 100, 120, 140, or 150 μm, wherein the lower limit is less than the upper limit.

In some embodiments, the diameter of the microcarrier is about 180 μm, about 160 μm, about 140 μm, about 120 μm, about 100 μm, about 90 μm, about 80 μm, about 70 μm, about 60 μm, about 50 μm, about 40 μm, about 30 μm, about 20 μm, or about 10 μm. In certain embodiments, the microcarrier is about 40 μm in diameter.

In some embodiments, the microcarrier is less than about 50 μm in thickness. For example, in some embodiments, the thickness of the microcarrier is less than about 70 μm, about 60 μm, about 50 μm, about 40 μm, about 30 μm, less than about 25 μm, less than about 20 μm, less than about 15 μm, less than about 10 μm, or less than about 5 μm. In some embodiments, the thickness of the microcarrier is less than about any of the following thicknesses (in μm): 50, 45, 40, 35, 30, 25, 20, 15, 10, 9, 8, 7, 6, 5, 4, 3, or 2. In some embodiments, the thickness of the microcarrier is greater than about any of the following thicknesses (in μm): 1, 2, 3, 4, 5, 6, 7, 8, 9, 10, 15, 20, 25, 30, 35, 40, or 45. That is, the thickness of the microcarrier may be any of a range of thicknesses (in μm) having an upper limit of 50, 45, 40, 35, 30, 25, 20, 15, 10, 9, 8, 7, 6, 5, 4, 3, or 2 and an independently selected lower limit of 1, 2, 3, 4, 5, 6, 7, 8, 9, 10, 15, 20, 25, 30, 35, 40, or 45, wherein the lower limit is less than the upper limit.

In some embodiments, the thickness of the microcarrier is about 50 μm, about 45 μm, about 40 μm, about 35 μm, about 30 μm, about 25 μm, about 20 μm, about 19 μm, about 18 μm, about 17 μm, about 16 μm, about 15 μm, about 14 μm, about 13 μm, about 12 μm, about 11 μm, about 10 μm, about 9 μm, about 8 μm, about 7 μm, about 6 μm, about 5 μm, about 4 μm, about 3 μm, about 2 μm, or about 1 μm. In some embodiments, the thickness of the microcarrier is between about 50 μm and about 2 μm, between about 20 μm and about 2 μm, or between about 10 μm and about 2 μm. In certain embodiments, the microcarrier is about 5 μm in thickness.

In some aspects, a microcarrier of the present disclosure can comprise a capture agent. In some embodiments, the capture agent for a particular microcarrier species may be a “unique capture agent,” e.g., a capture agent is associated with a particular microcarrier species having a particular identifier (e.g., analog code). The capture agent can be any biomolecule or a chemical compound capable of binding one or more analytes (such as a biomolecule or chemical compound) present in the solution. Examples of biomolecule capture agents comprise, but are not limited to, a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment. Examples of chemical compound capture agents comprise, but are not limited to, individual components of chemical libraries, small molecules, or environmental toxins (for example, pesticides or heavy metals).

In some embodiments, the capture agent is coupled to a surface of the microcarrier (in some embodiments, in at least a center portion of the microcarrier surface). In some embodiments, the capture agent can be chemically attached to the microcarrier. In other embodiments, the capture agent can be physically absorbed to the surface of the microcarrier. In some embodiments, the attachment linkage between the capture agent and the microcarrier surface can be a covalent bond. In other embodiments, the attachment linkage between the capture agent and the microcarrier surface can be a non-covalent bond comprising, but not limited to, a salt bridge or other ionic bond, one or more hydrogen bonds, hydrophobic interactions, Van der Waals force, London dispersion force, a mechanical bond, one or more halogen bonds, aurophilicity, intercalation, or stacking.

In some aspects, more than one (such as two, three, four, five, six, seven, eight, nine, or ten) capture agents for the same analyte can each be associated with a microcarrier described herein. In this embodiment, each capture agent for a particular analyte binds to the analyte with a different affinity as measured by the dissociation constant of analyte/capture agent binding. Accordingly, within a plurality of microcarriers in a composition, there can be two or more subpopulations of microcarriers with capture agents that bind to the same analyte, but wherein the capture agents associated with each subpopulation bind to the analyte with a different affinity. In some embodiments, the dissociation constant of the analyte for any of the capture agents is not greater than 10⁻⁶ M, such as 10⁻⁷ M or 10⁻⁸ M. In other embodiments, the dissociation constant of the analyte for any of the capture agents is from about 10⁻¹⁰ M to about 10⁻⁶ M, such from about 10⁻¹⁰ M to about 10⁻⁷ M, about 10^{-1°} M to about 10⁻⁸ M, about 10^{-1°} M to about 10⁻⁹ M, about 10⁻⁹ M to about 10⁻⁶ M, about 10⁻⁹ M to about 10⁻⁷ M, about 10⁻⁹ M to about 10⁻⁸ M, about 10⁻⁸ M to about 10⁻⁶ M, or about 10⁻⁸ M to about 10⁻⁷ M. In some embodiments, the dissociation constant of the analyte for any two capture agents differs by as much as about 3 log₁₀, such as by as much as about 2.5 log₁₀, 2 log₁₀, 1.5 log₁₀, or 1 log₁₀.

In some embodiments, an analyte of the present disclosure is coupled to a microcarrier for the capture of one or more analytes. In some embodiments, the one or more analytes may be captured from a sample, such as a biological sample described herein. In some embodiments, an analyte may comprise without limitation a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment. In other embodiments, the analyte is a chemical compound (such as a small molecule chemical compound) capable of binding to the capture agent such as individual components of chemical libraries, small molecules, or environmental toxins (for example, pesticides or heavy metals).

In some aspects, the analytes in a sample (such as a biological sample) can be labeled with a signal-emitting entity capable of emitting a detectable signal upon binding to the capture agent. In some embodiments, the signal-emitting entity can be colorimetric based. In other embodiments, the signal-emitting entity can be fluorescence-based comprising, but not limited to, phycoerythrin, blue fluorescent protein, green fluorescent protein, yellow fluorescent protein, cyan fluorescent protein, and derivatives thereof. In other embodiments, the signal-emitting entity can be radioisotope based, comprising, but not limited to, molecules labeled with ³²P, ³³P, ²²Na, ³⁶Cl, ²H , ³H, ³⁵S, and ¹²³I. In other embodiments, the signal-emitting entity is light-based comprising, but not limited to, luciferase (e.g., chemiluminescence-based), horseradish peroxidase, alkaline phosphatase, and derivatives thereof. In some embodiments, the biomolecules or chemical compounds present in the sample can be labeled with the signal-emitting entity prior to contact with the microcarrier. In other embodiments, the biomolecules or chemical compounds present in the sample can be labeled with the signal-emitting entity subsequent to contact with the microcarrier.

### IV. Methods of Making Encoded Microcarriers

Certain aspects of the present disclosure relate to methods for making an encoded microcarrier, e.g., a microcarrier described herein. The methods for making an encoded microcarrier may comprise one or more of the microcarrier features or aspects described herein, e.g., in section III above and/or the Examples that follow.

In some embodiments, the methods comprise depositing a substantially transparent magnetic polymer layer (e.g., comprising a mixture of a substantially transparent polymer and a plurality of magnetic nanoparticles, such as magnetic nanoparticles comprising iron(II,III) oxide or iron(III) oxide), patterning the deposited substantially transparent magnetic polymer layer into a microcarrier shape of the present disclosure, depositing a substantially non-transparent layer (e.g., onto at least a portion of the deposited substantially transparent magnetic polymer layer), and patterning the deposited substantially non-transparent layer into a two-dimensional shape representing an analog code of the present disclosure. For example, the substantially transparent magnetic polymer layer can be deposited onto a substrate of the present disclosure.

In some embodiments, the methods comprise (a) depositing a substantially transparent magnetic polymer layer having a first surface and a second surface, the first and the second surfaces being parallel to each other, wherein the substantially transparent magnetic polymer layer comprises a mixture of a substantially transparent polymer and a plurality of magnetic nanoparticles, and wherein the magnetic nanoparticles comprise iron(II,III) oxide or iron(III) oxide; (b) patterning the deposited substantially transparent magnetic polymer layer into a microcarrier shape; (c) depositing a substantially non-transparent layer onto the first surface of the deposited substantially transparent magnetic polymer layer; and (d) patterning the deposited substantially non-transparent layer into a two-dimensional shape representing an analog code. In some variations, step (a) comprises depositing the substantially transparent magnetic polymer layer onto a sacrificial layer. In some variations, the deposited substantially non-transparent layer is patterned by lift-off process. In some variations, the method comprises, prior to (c) and after (a), depositing a second polymer layer onto the first surface of the deposited substantially transparent magnetic polymer layer; prior to (c) and after (a), applying a mask to the deposited second polymer layer, thereby generating a masked portion of the deposited second polymer layer and a non-masked portion of the deposited second polymer layer; prior to (c) and after (a), applying light to the masked, deposited second polymer layer, wherein the light is of a wavelength sufficient to remove the non-masked portion of the deposited second polymer layer; wherein (c) comprises depositing the substantially non-transparent layer onto the first surface of the deposited substantially transparent magnetic polymer layer and the masked portion of the deposited second polymer layer; and wherein (d) comprises removing the deposited second polymer layer, thereby patterning the deposited substantially non-transparent layer by removing the substantially non-transparent layer deposited onto the masked portion of the deposited second polymer layer.

In some embodiments, the methods comprise depositing a substantially non-transparent layer, patterning the deposited substantially non-transparent layer into a two-dimensional shape representing an analog code of the present disclosure, depositing (e.g., onto at least a portion of the deposited substantially non-transparent layer) a substantially transparent magnetic polymer layer (e.g., comprising a mixture of a substantially transparent polymer and a plurality of magnetic nanoparticles, such as magnetic nanoparticles comprising iron(II,III) oxide or iron(III) oxide), and patterning the deposited substantially transparent magnetic polymer layer into a microcarrier shape of the present disclosure. For example, the substantially non-transparent layer can be deposited onto a substrate of the present disclosure.

In some embodiments, a substantially transparent magnetic polymer layer or substantially non-transparent layer of the present disclosure may be deposited on a substrate. Suitable substrates may comprise substrates used in standard semiconductor and/or micro-electro-mechanical systems (MEMS) fabrication techniques. In some embodiments, the substrate may comprise glass, silicon, quartz, plastic, polyethylene terephthalate (PET), an indium tin oxide (ITO) coating, or the like.

In some embodiments, a sacrificial layer may be deposited on the substrate, e.g., a substrate or substrate carrier as described herein. In some embodiments, the sacrificial layer may be made of a polymer, comprising without limitation polyvinyl alcohol (PVA) or OmniCoat™ (MicroChem; Newton, MA). Sacrificial layers may be applied, used, and dissolved or stripped, e.g., according to manufacturer's instructions.

In some embodiments, a substantially transparent magnetic polymer layer or substantially non-transparent layer of the present disclosure is deposited on a sacrificial layer. To generate a planar microcarrier surface using a substantially transparent magnetic polymer layer, or a planar substantially non-transparent layer constituting a two-dimensional shape that represents an analog code, the corresponding layer may be deposited onto a planar sacrificial layer.

In some embodiments that employ an optional sacrificial layer and/or substrate/substrate carrier of the present disclosure, the sacrificial layer may be dissolved, etched out, or stripped, and/or the substrate/substrate carrier may be removed, using a solvent. A variety of solvents useful for fabrication (e.g., in standard semiconductor or MEMS fabrication processes, such as photoresist removal) are known in the art. In some embodiments, the solvent is a photoresist stripper solvent, such as a DMSO- or 1-methyl-2-pyrrolidon (NMP)-based solvent. In some embodiments, the solvent is an AZ® photoresist stripper, such as AZ® 300T (AZ Electronic Materials; Somerville, NJ).

In some embodiments, a deposited substantially non-transparent layer is patterned by lift-off process. In some embodiments, the substantially non-transparent layer is patterned by: depositing a second polymer layer (e.g., onto the substantially transparent magnetic polymer layer, if deposited; otherwise, onto a sacrificial layer); applying a mask to the deposited second polymer layer; applying light to the masked, deposited second polymer layer; depositing the substantially non-transparent layer onto the masked portion of the deposited second polymer layer (as well as onto the substantially transparent magnetic polymer layer, if deposited; otherwise, onto a sacrificial layer); and removing the deposited second polymer layer. In some embodiments, the light is of a wavelength sufficient to remove the non-masked portion of the deposited second polymer layer. Thus, the deposited substantially non-transparent layer is patterned by removing the substantially non-transparent layer deposited onto the masked portion of the deposited second polymer layer. In some embodiments, the second polymer layer is deposited by spin coating.

In some embodiments, the methods described herein for making an encoded microcarrier further comprise generating a mixture of monomer for a substantially transparent polymer and a plurality of magnetic nanoparticles (e.g., magnetic nanoparticles comprising iron(II,III) oxide or iron(III) oxide) for a substantially transparent magnetic polymer layer of the present disclosure. For example, the monomer for the substantially transparent polymer and the plurality of magnetic nanoparticles can be mixed by vortexing or sonication.

In some embodiments, prior to depositing a substantially transparent magnetic polymer layer, the method described herein for making an encoded microcarrier further comprises mixing monomer for a substantially transparent polymer, a plurality of magnetic nanoparticles, a solvent, and a dispersing agent to form a mixture of the monomer for the substantially transparent polymer, the plurality of magnetic nanoparticles, the solvent, and the dispersing agent. In some embodiments, the plurality of magnetic nanoparticles comprises 2.5% of the mixture of the monomer for the substantially transparent polymer, the plurality of magnetic nanoparticles, the solvent, and the dispersing agent. In some embodiments, the plurality of magnetic nanoparticles comprises less than about any of the following concentrations (% by weight with respect to the mixture of the monomer for the substantially transparent polymer, the plurality of magnetic nanoparticles, the solvent, and the dispersing agent): 10, 9, 8, 7, 6, 5, 4, 3, 2.5 2, 1.5, 1, 0.9, 0.8, 0.7, 0.6, 0.5, 0.4, or 0.3. In some embodiments, the plurality of magnetic nanoparticles comprises greater than about any of the following concentrations (% by weight with respect to the mixture of the monomer for the substantially transparent polymer, the plurality of magnetic nanoparticles, the solvent, and the dispersing agent): 0.1, 0.2, 0.3, 0.4, 0.5, 0.6, 0.7, 0.8, 0.9, 1, 1.5, 2, 2.5, 3, 4, 5, 6, 7, or 8. That is, the plurality of magnetic nanoparticles in the present disclosure can have a concentration with an upper limit of 10, 9, 8, 7, 6, 5, 4, 3, 2.5, 2, 1.5, 1, 0.9, 0.8, 0.7, 0.6, 0.5, 0.4, or 0.3% and an independently selected lower limit of 0.1, 0.2, 0.3, 0.4, 0.5, 0.6, 0.7, 0.8, 0.9, 1, 1.5, 2, 2.5, 3, 4, 5, 6, 7, or 8%, wherein the lower limit is less than the upper limit and the concentration is % by weight with respect to the mixture of the monomer for the substantially transparent polymer, the plurality of magnetic nanoparticles, the solvent, and the dispersing agent.

In some variations, a plurality of magnetic nanoparticles, a solvent, and a dispersing agent are mixed to form a mixture of the plurality of magnetic nanoparticles, the solvent, and the dispersing agent, prior to mixing with monomer for a substantially transparent polymer. In some embodiments, the dispersing agent comprises 10% of the mixture of the plurality of magnetic nanoparticles, the solvent, and the dispersing agent. In some embodiments, the dispersing agent comprises less than about any of the following concentrations (% by weight with respect to the mixture of the plurality of magnetic nanoparticles, the solvent, and the dispersing agent): 90, 80, 70, 60, 50, 40, 30, 20, 15, 14, 13, 12, 11, 10, 9, 8, 7, 6, 5, 4, 3, 2, 1, 0.5, or 0.1. In some embodiments, the plurality of magnetic nanoparticles comprises greater than about any of the following concentrations (% by weight with respect to the mixture of the plurality of magnetic nanoparticles, the solvent, and the dispersing agent): 0.1, 0.5, 1, 2, 3, 4, 5, 6, 7, 8, 9, 10, 11, 12, 13, 14, 15, 20, 30, 40, 50, 60, 70, 80, or 90. That is, the plurality of magnetic nanoparticles in the present disclosure can have a concentration with an upper limit of 90, 80, 70, 60, 50, 40, 30, 20, 15, 14, 13, 12, 11, 10, 9, 8, 7, 6, 5, 4, 3, 2, 1, 0.5, or 0.1% and an independently selected lower limit of 0.1, 0.5, 1, 2, 3, 4, 5, 6, 7, 8, 9, 10, 11, 12, 13, 14, 15, 20, 30, 40, 50, 60, 70, 80, or 90%, wherein the lower limit is less than the upper limit and the concentration is % by weight with respect to the mixture of the plurality of magnetic nanoparticles, the solvent, and the dispersing agent.

In some embodiments, the solvent described herein comprises cyclopentanone or g-Butyrolactone, or a combination thereof.

In some embodiments, the dispersing agent described herein comprises phosphoric acid polymer or contains a phosphate group. In some embodiments, the dispersing agent comprises polyethylene glycol (PEG) polymers. In some embodiments, the dispersing agent comprises polyester polymers. In some embodiments, the dispersing agent comprises phosphoric acid polymers. In some embodiments, the dispersing agent comprises polymers with one or more carboxyl groups. In some embodiments, the dispersing agent comprises polymers with one or more phosphate groups. In some embodiments, the dispersing agent comprises polymers with PEG-phosphoric acid moiety. In some embodiments, the dispersing agent comprises carboxyl-PEG-phosphoric acid, e.g., as described in J. Mater. Chem., 2021, 22, 19806. In some embodiments, the dispersing agent comprises phosphoric acid polyester. In some embodiments, 100% of the dispersing agent is carboxyl-PEG-phosphoric acid. In some embodiments, 90%, 80%, 70%, 60%, 50%, 40%, 30%, 20%, 10%, 5%, 1%, or 0.1% of the dispersing agent is carboxyl-PEG-phosphoric acid. In some embodiments, dispersing agent allows magnetic nanoparticles and monomers for a substantially transparent polymer in a mixture to remain stable, thereby reducing lot-to-lot variations in the microcarriers produced. In some embodiments, mixing magnetic nanoparticles with a solvent comprising phosphate polyester polymers as dispersing agent enables the mixture comprising the magnetic nanoparticles and monomers for a substantially transparent polymer to remain stable for greater than about any of the following days: 5, 6, 7, 8, 9, 10, 15, 20, 25, 30, 35, 40, 45, or 50.

In some embodiments, a substantially transparent magnetic polymer layer of the present disclosure is deposited by spin coating. In some embodiments, a substantially transparent magnetic polymer layer of the present disclosure is deposited by spray coating. In some embodiments, a substantially transparent magnetic polymer layer is deposited and/or patterned by photolithography. In some embodiments, a substantially transparent magnetic polymer layer is deposited and/or patterned by lift-off. In some embodiments, a substantially transparent magnetic polymer layer is deposited and/or patterned by sputtering.

In some embodiments, a substantially non-transparent layer of the present disclosure is deposited by lithography. In some embodiments, a substantially non-transparent layer of the present disclosure is deposited by lift-off.

In some embodiments, a capture agent may be coupled to a microcarrier of the present disclosure, e.g., a microcarrier described herein and/or a microcarrier produced by any of the methods described herein. Any of the capture agents described herein, or any capture agent known in the art suitable for capturing an analyte described herein, may find use in the methods and/or microcarriers of the present disclosure.

In some embodiments, the capture agent may be coupled to a polymer layer of the present disclosure, e.g., a substantially transparent magnetic polymer layer described herein. In some embodiments, the capture agent may be coupled to one or both of a first or a second surface of the polymer layer. In some embodiments, the capture agent may be coupled to at least the center portion of the polymer layer (e.g., a center portion as described herein). In some embodiments, the polymer comprises an epoxy-based polymer or otherwise contains an epoxide group.

In some embodiments, coupling the capture agent involves reacting the polymer with a photoacid generator and light to generate a cross-linked polymer. In some embodiments, the light is of a wavelength that activates the photoacid generator, e.g., UV or near-UV light. Photoacid generators are commercially available from Sigma-Aldrich (St. Louis) and BASF (Ludwigshafen). Any suitable photoacid generator known in the art may be used, comprising without limitation triphenyl or triaryl sulfonium hexafluoroantimonate; triarylsulfonium hexafluorophosphate; triphenylsulfonium perfluoro-1-butanesulfonate; triphenylsulfonium triflate; Tris(4-tert-butylphenyl)sulfonium perfluoro-1-butanesulfonate or triflate; Bis(4-tert-butylphenyl)iodonium-containing photoacid generators such as Bis(4-tert-butylphenyl)iodonium perfluoro-1-butanesulfonate, p-toluenesulfonate, and triflate; Boc-methoxyphenyldiphenylsulfonium triflate; (tert-Butoxycarbonylmethoxynaphthyl)-diphenylsulfonium triflate; (4-tert-Butylphenyl)diphenylsulfonium triflate; diphenyliodonium hexafluorophosphate, nitrate, perfluoro-1-butanesulfonate, triflate, or p-toluenesulfonate; (4-fluorophenyl)diphenylsulfonium triflate; N-hydroxynaphthalimide triflate; N-hydroxy-5-norbornene-2,3-dicarboximide perfluoro-1-butanesulfonate; (4-iodophenyl)diphenylsulfonium triflate; (4-methoxyphenyl)diphenylsulfonium triflate; 2-(4-methoxystyryl)-4,6-bis(trichloromethyl)-1,3,5-triazine; (4-methylphenyl)diphenylsulfonium triflate; (4-methylthiophenyl)methyl phenyl sulfonium triflate; (4-phenoxyphenyl)diphenylsulfonium triflate; (4-phenylthiophenyl)diphenylsulfonium triflate; or any of the photoacid generators described in product-finder.basf.com/group/corporate/product-finder/de/literature-document:/Brand+Irgacure-Brochure--Photoacid+Generator+Selection+Guide-English.pdf. In some embodiments, the photoacid generator is a sulfonium-containing photoacid generator.

In some embodiments, coupling the capture agent involves reacting an epoxide of the cross-linked polymer with a functional group such as an amine, carboxyl, thiol, or the like. Alternatively, the epoxy group on the surface can be oxidized to hydroxyl group, which is subsequently used as initiation sites for graft polymerization of water soluble polymers such as poly(acrylic acid). The carboxyl groups in poly(acrylic acid) are then used to form covalent bonds with amino or hydroxyl groups in capture agents.

In some embodiments, coupling the capture agent involves reacting an epoxide of the cross-linked polymer with a compound that contains an amine and a carboxyl. In some embodiments, the amine of the compound reacts with the epoxide to form a compound-coupled, cross-linked polymer. Without wishing to be bound to theory, it is thought that the capture agent may be coupled to the polymer before the polymer is cross-linked; however, this may reduce the uniformity of the resulting surface. Any compound with a primary amine and a carboxyl group may be used. Compounds may comprise without limitation glycine, amino undecanoic acid, amino caproic acid, acrylic acid, 2-carboxyethyl acrylic acid, 4-vinylbenzoic acid, 3-acrylamido-3-methyl-1-butanoic acid, glycidyl methacrylate, and the like. In some embodiments, the carboxyl of the compound-coupled, cross-linked polymer reacts with an amine (e.g., a primary amine) of the capture agent to couple the capture agent to the substantially transparent polymer.

Descriptions of various capture agents and analytes suitable for the methods described above may be found throughout the present disclosure, e.g., in section III above.

### V. Multiplex Assays

Certain aspects of the present disclosure relate to methods for detecting analytes in a solution by using an encoded microcarrier, e.g., a microcarrier described herein. The methods for analyte detection using an encoded microcarrier that comprises one or more of the microcarrier features or aspects described herein, e.g., in sections III and IV above and/or the Examples that follow. Advantageously, these encoded microcarriers allow for analyte detection in improved multiplex assays with a large number of potential unique microcarriers and reduced recognition error, as compared to traditional multiplex assays. The analyte detection methods used herein may be performed in any suitable assay vessel known in the art, for example a microplate, petri dish, or any number of other well-known assay vessels.

In some embodiments, the methods for detecting analytes in a solution comprise contacting a solution comprising a first analyte and a second analyte with a plurality of microcarriers, where the plurality of microcarriers comprises at least a first microcarrier of the present disclosure that specifically captures the first analyte and is encoded with a first analog code, and a second microcarrier of the present disclosure that specifically captures the second analyte and is encoded with a second analog code; decoding the first analog code and the second analog code using analog shape recognition to identify the first microcarrier and the second microcarrier; and detecting an amount of the first analyte bound to the first microcarrier and an amount of the second analyte bound to the second microcarrier.

In some embodiments, the methods comprise contacting a solution comprising a first analyte and a second analyte with a plurality of microcarriers. In some embodiments, the plurality of microcarriers may comprise a first microcarrier of the present disclosure that specifically captures the first analyte (e.g., using a capture agent, coupled to the microcarrier, specific for the first analyte), where the first microcarrier is encoded with a first analog code; and a second microcarrier of the present disclosure that specifically captures the second analyte (e.g., using a capture agent, coupled to the microcarrier, specific for the second analyte), where the second microcarrier is encoded with a second analog code different from the first analog code. In some embodiments, the first and second analytes may be different. In other embodiments, the first and second analytes may be the same, e.g., the first and second microcarriers may redundantly recognize the same analyte (this may be useful, e.g., for quality control purposes), or they may recognize distinct regions of the same analyte (e.g., antibodies recognizing different epitopes of the same antigen).

The methods of the present disclosure may be used to detect analytes in any suitable solution. In some embodiments, the solution comprises a biological sample. Examples of biological samples comprise without limitation blood, urine, sputum, bile, cerebrospinal fluid, interstitial fluid of skin or adipose tissue, saliva, tears, bronchial-alveolar lavage, oropharyngeal secretions, intestinal fluids, cervico-vaginal or uterine secretions, and seminal fluid. In some embodiments, the biological sample may be from a human. In other embodiments, the solution comprises a sample that is not a biological sample, such as an environmental sample, a sample prepared in a laboratory (e.g., a sample containing one or more analytes that have been prepared, isolated, purified, and/or synthesized), a fixed sample (e.g., a formalin-fixed, paraffin-embedded or FFPE sample), and so forth.

In some embodiments, the analysis is multiplexed, that is, each solution (e.g., a sample) is analyzed so that a signal from the signal emitting entity is detected by the reaction detection system for at least 2 analytes of interest, at least 3 analytes of interest, at least 4 analytes of interest, at least 5 analytes of interest, at least 10 analytes of interest, at least 15 analytes of interest, at least 20 analytes of interest, at least 25 analytes of interest, at least 30 analytes of interest, at least 35 analytes of interest, at least 40 analytes of interest, at least 45 analytes of interest, or at least 50 analytes of interest, or more.

In some embodiments, the methods comprise decoding the first analog code and the second analog code using analog shape recognition to identify the first microcarrier and the second microcarrier. Conceptually, this decoding may involve imaging the analog code of each microcarrier (e.g., in a solution or sample), comparing each image against a library of analog codes, and matching each image to an image from the library, thus positively identifying the code. Optionally, as described herein, when using microcarriers that comprise an orientation indicator (e.g., an asymmetry), the decoding may further comprise a step of rotating each image to align with a particular orientation (based in part, e.g., on the orientation indicator). For example, if the orientation indicator comprises a gap, the image could be rotated until the gap reaches a predetermined position or orientation (e.g., a 0° position of the image).

Various shape recognition software, tools, and methods are known in the art. Examples of such APIs and tools comprise without limitation Microsoft® Research FaceSDK, OpenBR, Face and Scene Recognition from ReKognition, Betaface API, and various ImageJ plugins. In some embodiments, the analog shape recognition may comprise without limitation image processing steps such as foreground extraction, shape detection, thresholding (e.g., automated or manual image thresholding), and the like.

It will be appreciated by one of skill in the art that the methods and microcarriers described herein may be adapted for various imaging devices, comprising without limitation a microscope, plate reader, and the like. In some embodiments, decoding the analog codes may comprise illuminating the first and second microcarriers by passing light through the substantially transparent portions (e.g., substantially transparent polymer layer(s)) of the first and second microcarriers and/or the surrounding solution. The light may then fail to pass through, or pass through with a lower intensity or other appreciable difference, the substantially non-transparent portions (e.g., substantially non-transparent polymer layer(s)) of the first and second microcarriers to generate a first analog-coded light pattern corresponding to the first microcarrier and a second analog-coded light pattern corresponding to the second microcarrier.

As described supra, any type of light microscopy may be used for the methods of the present disclosure, comprising without limitation one or more of: bright field, dark field, phase contrast, differential interference contrast (DIC), Nomarski interference contrast (NIC), Nomarski, Hoffman modulation contrast (HMC), or fluorescence microscopy. In certain embodiments, the analog codes may be decoded using bright field microscopy, and analyte(s) may be detected using fluorescence microscopy.

In some embodiments, decoding the analog codes may further comprise imaging the first analog-coded light pattern to generate a first analog-coded image and imaging the second analog-coded light pattern to generate a second analog-coded image. That is to say, the pattern of imaged light may correspond to the pattern of substantially transparent/substantially non-transparent areas of the microcarrier, thus producing an image of the analog codes. This imaging may comprise steps comprising without limitation capturing the image, thresholding the image, and any other image processing step desired to achieve more accurate, precise, or robust imaging of the analog codes.

In some embodiments, decoding the analog codes may further comprise using analog shape recognition to match the first analog-coded image with the first analog code and to match the second analog-coded image with the second analog code. In some embodiments, an image may be matched with an analog code (e.g., an image file from a library of image files, with each image file corresponding to a unique two-dimensional shape/analog code) within a predetermined threshold, e.g., that tolerates a predetermined amount of deviation or mismatch between the image and the exemplar analog code image. Such a threshold may be empirically determined and may naturally be based on the particular type of two-dimensional shapes used for the analog codes and the extent of variation among the set of potential two-dimensional shapes.

In some embodiments, the methods comprise detecting an amount of the first analyte bound to the first microcarrier and an amount of the second analyte bound to the second microcarrier. Any suitable analyte detection technique(s) known in the art may be used. For example, in some embodiments, the first and the second microcarriers may be incubated with one or more detection agents. In some embodiments, the one or more detection agents bind the first analyte captured by the first microcarrier and the second analyte captured by the second microcarrier. In some embodiments, the methods further comprise measuring the amount of detection agent bound to the first and the second microcarriers.

In some embodiments, the analytes in a solution (such as a biological sample) can be labeled with a detection agent (e.g., a signal-emitting entity) capable of emitting a detectable signal upon binding to the capture agent. In some embodiments, the detection agent can be colorimetric based. In other embodiments, the detection agent can be fluorescence-based comprising, but not limited to, phycoerythrin, blue fluorescent protein, green fluorescent protein, yellow fluorescent protein, cyan fluorescent protein, and derivatives thereof. In other embodiments, the detection agent can be radioisotope based, comprising, but not limited to, molecules labeled with ³²P, ³³P, ²²Na, ³⁶Cl, ²H, ³H, ³⁵S, and ¹²³I. In other embodiments, the detection agent is light-based comprising, but not limited to, luciferase (e.g. chemiluminescence-based), horseradish peroxidase, alkaline phosphatase and derivatives thereof. In some embodiments, the biomolecules or chemical compounds present in the solution can be labeled with the detection agent prior to contact with the microcarrier composition. In other embodiments, the biomolecules or chemical compounds present in the solution can be labeled with the detection agent subsequent to contact with the microcarrier composition. In yet other embodiments, the detection agent may be coupled to a molecule or macromolecular structure that specifically binds the analyte of interest, e.g., a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and/or an antibody fragment.

In some embodiments, the detection agent is a fluorescent detection agent, and the amount of detection agent bound to the first and the second microcarriers is measured by fluorescence microscopy (e.g., a fluorescent microscope or plate reader). In other embodiments, the detection agent is a luminescent detection agent, and the amount of detection agent bound to the first and the second microcarriers is measured by luminescence microscopy (e.g., a luminescent microscope or plate reader).

In some embodiments, each analyte/capture agent may be used with a specific detection agent. As non-limiting examples, the detection agent may be a detection agent (e.g., a fluorescent, luminescent, enzymatic, or other detection agent) coupled to an antibody that specifically binds the analyte; or a ligand or receptor of a ligand-receptor pair, if the analyte is a cognate ligand/receptor of the ligand-receptor pair. This technique is conceptually similar to a sandwich ELISA or protein microarray that comprises a capture and a detection antibody (though it should be noted in the present case that the agents in this example are not strictly limited to antibodies). As another non-limiting example, the detection agent may be a fluorescent or other detectable probe coupled to a protein of interest, such as a labeled analyte of interest. For example, a reaction may be used to couple detection agent(s) to one or more proteins in a solution of interest (e.g., a sample), which would then be captured by the capture agents (conceptually similar to an antigen capture-type of protein microarray).

In other embodiments, multiple unique analytes/capture agents may be used with a universal detection agent. As non-limiting examples, the detection agent may be an agent that binds to the Fc region of an antibody, if the analyte is an antibody; a fluorescent or other detectable probe coupled to an oligonucleotide (e.g., a single stranded oligonucleotide that hybridizes with an analyte), if the analyte is a polynucleotide such as DNA or RNA. The later scenario is conceptually similar to a microarray technique.

In some embodiments, the detecting steps may comprise one or more washing steps, e.g., to reduce contaminants, remove any substances non-specifically bound to the capture agent and/or microcarrier surface, and so forth. In some embodiments, a magnetic separation step may be used to wash a microcarrier containing a magnetic layer or material of the present disclosure. In other embodiments, other separation steps known in the art may be used.

In some embodiments, the decoding step(s) may occur after the detecting step(s). In other embodiments, the decoding step(s) may occur before the detecting step(s). In still other embodiments, the decoding step(s) may occur simultaneously with the detecting step(s).

### VI. Kits or Articles of Manufacture

Further provided herein are kits or articles of manufacture containing a plurality of microcarriers of the present disclosure. These kits or articles of manufacture may find use, inter alia, in conducting a multiplex assay, such as the exemplary multiplex assays described herein (see, e.g., section V above).

In some embodiments, the kits or articles of manufacture may comprise a first microcarrier of the present disclosure that specifically captures a first analyte (e.g., using a capture agent, coupled to the microcarrier, specific for the first analyte), where the first microcarrier is encoded with a first analog code; and a second microcarrier of the present disclosure that specifically captures the second analyte (e.g., using a capture agent, coupled to the microcarrier, specific for the second analyte), where the second microcarrier is encoded with a second analog code different from the first analog code. In some embodiments, the first and second analytes may be different. In other embodiments, the first and second analytes may be the same, e.g., the first and second microcarriers may redundantly recognize the same analyte (this may be useful, e.g., for quality control purposes), or they may recognize distinct regions of the same analyte (e.g., antibodies recognizing different epitopes of the same antigen). The kits or articles of manufacture may comprise any of the microcarriers described herein (see, e.g., section III above) or produced using the methods described herein (see, e.g., section IV above).

In some embodiments, the kits or articles of manufacture may further comprise one or more detection agents of the present disclosure for detecting an amount of the first analyte bound to the first microcarrier and an amount of the second analyte bound to the second microcarrier. In some embodiments, the detection agent for the first analyte may be the same as the detection agent for the second analyte. In other embodiments, the detection agent for the first analyte may be different from the detection agent for the second analyte.

In some embodiments, the kits or articles of manufacture may further comprise instructions for using the kit or articles of manufacture to detect one or more analytes, e.g., the first and the second analyte. These instructions may be for using the kit or article of manufacture, e.g., in any of the methods described herein.

In some embodiments, the kits or articles of manufacture may further comprise one or more detection agents (e.g., as described above), along with any instructions or reagents suitable for coupling a detection agent to one or more analytes, or for coupling a detection agent to one or more macromolecules that recognize an analyte. The kits or articles of manufacture may further comprise any additional components for using the microcarriers in an assay (e.g., a multiplex assay), comprising without limitation a plate (e.g., a 96-well or other similar microplate), dish, microscope slide, or other suitable assay container; a non-transitory computer-readable storage medium (e.g., containing software and/or other instructions for analog shape or code recognition); washing agents; buffers; plate sealers; mixing containers; diluents or storage solutions; and the like.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be comprised within the spirit and purview of this application and scope of the appended claims.

Attention is now directed towards microcarriers for multiplex assays (e.g., analyte detection) and their methods of production. The following Examples illustrate exemplary embodiments of analog-encoded microcarriers for analyte detection that may find use, inter alia, in the methods, assays, and kits or articles of manufacture described herein. It is to be noted that these exemplary embodiments are in no way intended to be limiting but are provided to illustrate some of the aspects and features set forth herein.

### Example 1: Encoded Microcarriers with a Two-Dimensional, Analog Code

As described above, analog-encoded microcarriers are highly advantageous for multiplexed assays due to the vast number of potential unique identifiers and reduced recognition error. This Example describes various types of microcarriers encoded with a two-dimensional shape, which may be used as an analog code for identification. It is to be understood that the encoded microcarriers of the present disclosure may comprise some or all of the optional features set forth below in any combination.

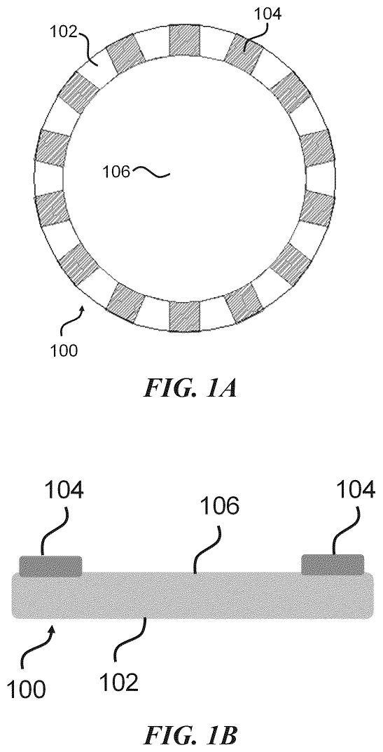

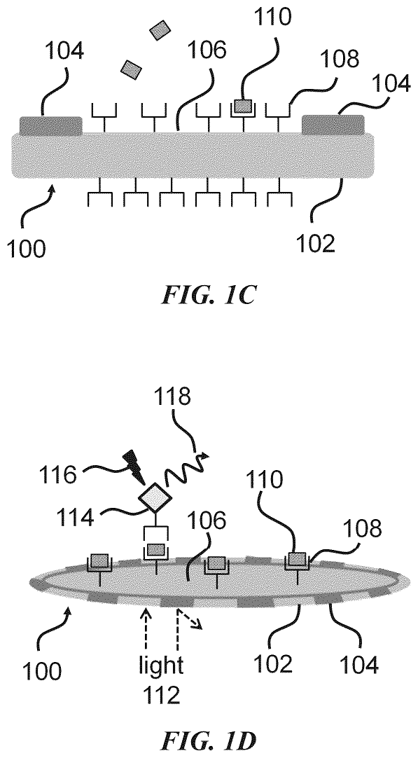

FIGS. 1A & 1B show two views of exemplary microcarrier 100. Microcarrier 100 is a circular disc of approximately 40 μm in diameter and 2-10 μm in thickness. FIG. 1A provides a view of microcarrier 100 looking at a circular face of the disc, while FIG. 1B shows a side view of microcarrier 100 orthogonal to the surface shown in FIG. 1A. Two components of microcarrier 100 are shown. First, substantially transparent polymer layer 102 provides the body of the microcarrier. Layer 102 may be produced, e.g., using a polymer such as SU-8, as described above. In some embodiments, layer 102 is a substantially transparent magnetic polymer, e.g., as described above.

Substantially non-transparent layer 104 is affixed to a surface of layer 102. While the cross-section of microcarrier 100 shown in FIG. 1B shows a discontinuous view of layer 104, the view shown in FIG. 1A illustrates that layer 104 is shaped like a circular gear with a plurality of teeth. This is merely an exemplary shape; other shapes are described above and shown, e.g., in FIGS. 2A-2C. Advantageously, the shape of layer 104 fits within the perimeter of layer 102. This allows for a variety of analog codes, each representing a unique identifier for one species of microcarrier, while maintaining a uniform overall shape across multiple species of microcarrier. Stated another way, each microcarrier species within a population of multiple species may have a different two-dimensional gear shape (i.e., analog code), but each microcarrier will have the same perimeter, leading to greater uniformity of physical properties (e.g., size, shape, behavior in solution, and the like). Layer 104 may be produced, e.g., using a polymer such as SU-8 mixed with a dye, a black matrix resist, or a metal that lacks residual magnetism, as described above.

Layer 104 surrounds center portion 106 of layer 102. A capture agent for capturing an analyte is coupled to at least center portion 106 on one or both surfaces (i.e., upper/lower surfaces) of layer 102. Advantageously, this allows center portion 106 to be imaged without any potential for interference resulting from layer 104.

FIGS. 1C & 1D show an exemplary assay using microcarrier 100 for analyte detection. FIG. 1C shows that microcarrier 100 may comprise capture agent 108 coupled to one or more surfaces in at least center portion 106. Microcarrier 100 is contacted with a solution containing analyte 110, which is captured by capture agent 108. As described above, various capture agents may be used to capture different types of analytes, ranging from small molecules, nucleic acids, and proteins (e.g., antibodies) to organelles, viruses, and cells. FIG. 1C illustrates a single microcarrier species (i.e., microcarrier 100), which captures analyte 110, but in a multiplex assay multiple microcarrier species are used, each species having a particular capture agent that recognizes a specific analyte.

FIG. 1D illustrates an exemplary process for “reading” microcarrier 100. This process comprises two steps that may be accomplished simultaneously or separately. First, the capture of analyte 110 by capture agent 108 is detected. In the example shown in FIG. 1D, detection agent 114 binds to analyte 110. Analyte not captured by a capture agent coupled to microcarrier 100 may have been washed off prior to detection, such that only analytes bound to microcarrier 100 are detected. Detection agent 114 also comprises a reagent for detection. As one example, detection agent 114 may comprise a fluorophore that, when excited by light 116 at a wavelength within the excitation spectrum of the fluorophore, emits light 118 (e.g., a photon). Light 118 may be detected by any suitable detection means, such as a fluorescence microscope, plate reader, and the like.

In addition, microcarrier 100 is read for its unique identifier. In the example shown in FIG. 1D, light 112 is used to illuminate the field containing microcarrier 100 (in some embodiments, light 112 may have a different wavelength than lights 116 and 118). When light 112 illuminates the field containing microcarrier 100, it passes through substantially transparent polymer layer 102 but is blocked by substantially non-transparent polymer layer 104, as shown in FIG. 1D. This generates a light pattern that can be imaged, for example, by light microscopy (e.g., using differential interference contrast, or DIC, microscopy). This light pattern is based on the two-dimensional shape (i.e., analog code) of microcarrier 100. Standard image recognition techniques may be used to decode the analog code represented by the image of microcarrier 100.

The analyte detection and identifier imaging steps may occur in any order, or simultaneously. Advantageously, both detection steps shown in FIG. 1D may be accomplished on one imaging device. As one example, a microscope capable of both fluorescence and light (e.g., bright field) microscopy may be used to quantify the amount of analyte 110 bound to microcarrier 100 (e.g., as detected by detection agent 114) and image the analog code created by layers 102 and 104. This allows for a more efficient assay process with fewer equipment requirements.

FIG. 2A illustrates three embodiments of an exemplary, gear-shaped coding scheme: microcarriers 200, 202, and 204. The unique codes of microcarriers 200, 202, and 204 are generated using a simple “filled or not filled” scheme. FIG. 2B illustrates 10 exemplary embodiments of a code using number of shapes (e.g., two distinct shapes in code ZN_3, as compared to seven distinct shapes in code ZN_10) and/or size of shapes (e.g., large, small, and intermediate-sized shapes in code ZN_2).

FIG. 2C shows another code format that uses a continuous or discontinuous ring shape in which the code can be expressed by any overall difference(s) in a number, pattern, two-dimensional width, and/or thickness in the overall shape (e.g., made up of one or more arc elements). The discontinuity in the ring shape provides an asymmetry that can be used, e.g., to orient an image of the code as part of decoding. Importantly, as described above, more complex encoding schemes are available using analog image recognition, thereby greatly expanding the number of potential unique codes.

### Example 2: Methods of Producing Microcarriers with a Two-Dimensional, Analog Code

Having described exemplary embodiments of microcarriers in the Example 1, attention is now directed to methods of producing microcarriers. As described above, the microcarriers of the present disclosure may be made of one, two, or more constituent layers, depending on the desired configuration and/or optional features.

FIG. 3 illustrates exemplary process 300 for producing a microcarrier of the present disclosure with a substantially transparent magnetic polymer layer and a substantially non-transparent layer.

At 302, substantially transparent magnetic polymer layer 304 was deposited onto sacrificial layer 306. In some embodiments, layer 304 is deposited by spin coating. Layer 304 can be made from any substantially transparent polymer with a superparamagnetic element, e.g., magnetic nanoparticles.

At 312, deposited layer 304 was patterned into a microcarrier shape, producing microcarrier shape 308b (also shown are partial views of microcarrier shapes 308a and 308c). That is, deposited layer 304 was patterned to produce a plurality of microcarrier shapes, with each shape comprising a substantially transparent magnetic polymer layer patterned into the microcarrier shape. In some embodiments, layer 304 is patterned using photo-lithography. In some embodiments, layer 304 is patterned using a lift-off process.

At 322, a substantially non-transparent layer was deposited and patterned onto each microcarrier (e.g., onto deposited and patterned substantially transparent magnetic polymer layers 308a, 308b, and 308c). In this view, substantially non-transparent layer 310a was deposited and patterned onto substantially transparent magnetic polymer layer 308a, substantially non-transparent layer 310b was deposited and patterned onto substantially transparent magnetic polymer layer 308b (layer 310b looks discontinuous in this view due to the cross-section, but can be either continuous or discontinuous), and substantially non-transparent layer 310c was deposited and patterned onto substantially transparent magnetic polymer layer 308c. Layers 310a, 310b, and 310c were patterned into an analog code, such as the exemplary types of analog codes shown in FIGS. 2A-2C. In this example, layers 310a, 310b, and 310c were deposited and patterned into a plurality of analog code shapes, with each analog code shape belonging to a microcarrier. The analog shapes can be the same or different from each other (e.g., sacrificial layer 306 can contain all microcarriers of one shape manufactured at the same time for convenience, or microcarriers representing two or more different analog shapes). In some embodiments, layers 310a, 310b, and 310c are deposited by lithography. In some embodiments, layers 310a, 310b, and 310c are patterned by lift-off process. Layers 310a, 310b, and 310c can be made from any non-transparent material, such as a substantially non-transparent polymer (e.g., comprising a black matrix resist or a polymer that exhibits an absorbance of greater than about 1.8 (OD) at a wavelength between about 230 nm and about 660 nm) or metal (e.g., lacking residual magnetism, such as titanium or chromium).

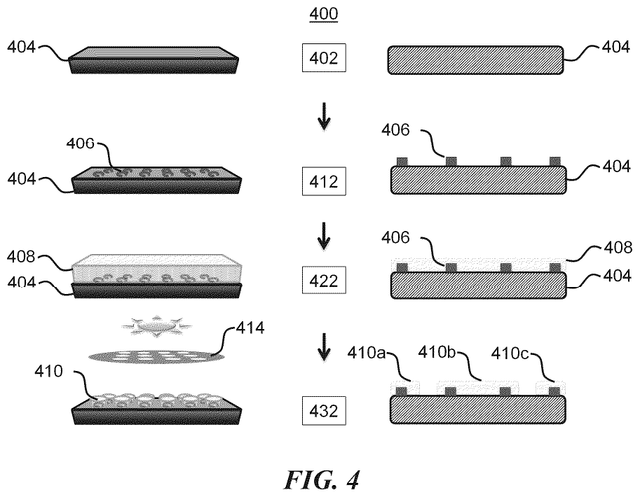

Alternatively, the orders of depositing and patterning the substantially transparent magnetic polymer and substantially non-transparent layers can be reversed, as shown in FIG. 4. FIG. 4 shows process 400 for producing a microcarrier of the present disclosure with a substantially transparent magnetic polymer layer and a substantially non-transparent layer.

At 402, sacrificial layer 404 is shown.

At 412, substantially non-transparent layer 406 was deposited onto sacrificial layer 404 and patterned. Layer 406 was patterned into an analog code, such as the exemplary types of analog codes shown in FIGS. 2A-2C. In this example, layer 406 was deposited and patterned into a plurality of analog code shapes, with each analog code shape belonging to a microcarrier. The analog shapes can be the same or different from each other (e.g., sacrificial layer 404 can contain all microcarriers of one shape manufactured at the same time for convenience, or microcarriers representing two or more different analog shapes). In some embodiments, layer 406 is deposited by lithography. In some embodiments, layer 406 is patterned by lift-off process. Layer 406 can be made from any non-transparent material, such as a substantially non-transparent polymer (e.g., comprising a black matrix resist or a polymer that exhibits an absorbance of greater than about 1.8 (OD) at a wavelength between about 230 nm and about 660 nm) or metal (e.g., lacking residual magnetism, such as titanium or chromium).

At 422, substantially transparent magnetic polymer layer 408 was deposited onto layer 406 and sacrificial layer 404. In some embodiments, layer 408 is deposited by spin coating. Layer 408 can be made from any substantially transparent polymer with a superparamagnetic element, e.g., magnetic nanoparticles.

At 432, layer 408 was patterned into microcarrier shape 410, producing microcarrier shapes 410a, 410b, and 410c. That is, deposited layer 408 was patterned to produce a plurality of microcarrier shapes, with each shape comprising a substantially transparent magnetic polymer layer patterned into the microcarrier shape. In some embodiments, layer 408 is patterned using photo-lithography, e.g., using mask 414. In some embodiments, layer 304 is patterned using a lift-off process.

Optionally, after 322 and/or 432, sacrificial layers 306 and/or 404 can be removed, e.g., by etching out.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. An encoded microcarrier, comprising:
(a) a substantially transparent magnetic polymer layer having a first surface and a second surface, the first and the second surfaces being parallel to each other, wherein the substantially transparent magnetic polymer comprises a mixture of a substantially transparent polymer and a plurality of magnetic nanoparticles, and wherein the magnetic nanoparticles comprise iron(II,III) oxide or iron(III) oxide;
(b) a substantially non-transparent layer, wherein the substantially non-transparent layer is affixed to the first surface of the substantially transparent magnetic polymer layer, and wherein an outline of the substantially non-transparent layer constitutes a two-dimensional shape representing an analog code; and
(c) a capture agent for capturing an analyte, wherein the capture agent is coupled to at least one of the first surface and the second surface of the substantially transparent magnetic polymer layer.

2. The microcarrier of claim 1, wherein the magnetic nanoparticles are superparamagnetic.

3. The microcarrier of claim 1 or claim 2, wherein the magnetic nanoparticles are less than about 30 nm in diameter and greater than or equal to about 3 nm in diameter.

4. The microcarrier of any one of claims 1-3, wherein the plurality of magnetic nanoparticles comprises less than about 10% (by weight) and greater than about 0.1% (by weight) of the mixture of the substantially transparent polymer and the plurality of magnetic nanoparticles.

5. The microcarrier of any one of claims 1-4, wherein the substantially transparent magnetic polymer layer is between about 0.1 μm and about 50 μm in thickness.

6. The microcarrier of any one of claims 1-5, wherein the substantially transparent polymer is an epoxy-based polymer.

7. The microcarrier of claim 6, wherein the epoxy-based polymer is SU-8.

8. The microcarrier of any one of claims 1-7, wherein the substantially non-transparent layer comprises a substantially non-transparent polymer.

9. The microcarrier of claim 8, wherein the substantially non-transparent layer comprises a black matrix resist.

10. The microcarrier of claim 8, wherein the substantially non-transparent polymer exhibits an absorbance of greater than about 1.8 (OD) at a wavelength between about 230 nm and about 660 nm.

11. The microcarrier of any one of claims 1-7, wherein the substantially non-transparent layer comprises a metal that lacks residual magnetism.

12. The microcarrier of claim 11, wherein the substantially non-transparent layer comprises titanium or chromium.

13. The microcarrier of any one of claims 1-12, wherein the substantially non-transparent layer is between about 0.05 μm and about 2 μm in thickness.

14. The microcarrier of any one of claims 1-13, wherein the analog code comprises one or more overlapping or partially overlapping arc elements forming a continuous or discontinuous ring.

15. The microcarrier of any one of claims 1-14, further comprising an orientation indicator for orienting the analog code of the substantially non-transparent layer.

16. The microcarrier of claim 15, wherein the orientation indicator comprises an asymmetry of the substantially non-transparent layer.

17. The microcarrier of any one of claims 1-16, wherein the microcarrier is a substantially circular disc.

18. The microcarrier of any one of claims 1-17, wherein the microcarrier is between about 5 μm and about 200 μm in diameter.

19. The microcarrier of claim 18, wherein the microcarrier is about 40 μm in diameter.

20. The microcarrier of any one of claims 1-19, wherein the microcarrier is less than about 50 μm in thickness.

21. The microcarrier of claim 20, wherein the microcarrier is between about 2 μm and about 10 μm in thickness.

22. The microcarrier of claim 21, wherein the microcarrier is about 5 μm in thickness.

23. The microcarrier of any one of claims 1-22, wherein the analyte is selected from the group consisting of a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment.

24. The microcarrier of any one of claims 1-23, wherein the capture agent for capturing the analyte is selected from the group consisting of a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment.

25. A method of making an encoded microcarrier, comprising:
(a) depositing a substantially non-transparent layer having a first surface and a second surface, the first and the second surfaces being parallel to each other;
(b) patterning the deposited substantially non-transparent layer into a two-dimensional shape representing an analog code;
(c) depositing a substantially transparent magnetic polymer layer onto the first surface of the deposited substantially non-transparent layer, wherein the substantially transparent magnetic polymer layer comprises a mixture of a substantially transparent polymer and a plurality of magnetic nanoparticles, and wherein the magnetic nanoparticles comprise iron(II,III) oxide or iron(III) oxide; and
(d) patterning the deposited substantially transparent magnetic polymer layer into a microcarrier shape.

26. The method of claim 25, wherein (a) comprises depositing the substantially non-transparent layer onto a sacrificial layer.

27. The method of claim 26, wherein the substantially non-transparent layer is deposited and patterned by lithography.

28. The method of claim 26, wherein the deposited substantially non-transparent layer is deposited and patterned by lift-off process.

29. The method of claim 28, further comprising:
prior to (a), depositing a second polymer layer onto the sacrificial layer;
prior to (a), applying a mask to the deposited second polymer layer, thereby generating a masked portion of the deposited second polymer layer and a non-masked portion of the deposited second polymer layer;
prior to (a), applying light to the masked, deposited second polymer layer, wherein the light is of a wavelength sufficient to remove the non-masked portion of the deposited second polymer layer;
wherein (a) comprises depositing the substantially non-transparent layer onto the sacrificial layer and the masked portion of the deposited second polymer layer; and
wherein (b) comprises removing the deposited second polymer layer, thereby patterning the deposited substantially non-transparent layer by removing the substantially non-transparent layer deposited onto the masked portion of the deposited second polymer layer.

30. The method of claim 29, wherein the second polymer layer is deposited by spin coating.

31. The method of any one of claims 26-30, further comprising, prior to (a)-(d), depositing the sacrificial layer onto a substrate carrier.

32. The method of any one of claims 26-31, further comprising, after (a)-(d), etching out the sacrificial layer.

33. The method of any one of claims 25-32, further comprising, prior to (c), mixing monomer for the substantially transparent polymer, the plurality of magnetic nanoparticles, a solvent, and a dispersing agent to form a mixture of the monomer for the substantially transparent polymer, the plurality of magnetic nanoparticles, the solvent, and the dispersing agent.

34. The method of claim 33, wherein the plurality of magnetic nanoparticles, the solvent, and the dispersing agent are mixed to form a mixture of the plurality of magnetic nanoparticles, the solvent, and the dispersing agent, prior to mixing with the monomer for the substantially transparent polymer.

35. The method of claim 34, wherein the dispersing agent comprises 10% of the mixture of the plurality of magnetic nanoparticles, the solvent, and the dispersing agent.

36. The method of any one of claims 33-35, wherein the plurality of magnetic nanoparticles comprises 2.5% of the mixture of the monomer for the substantially transparent polymer, the plurality of magnetic nanoparticles, the solvent, and the dispersing agent.

37. The method of any one of claims 33-36, wherein the solvent comprises cyclopentanone.

38. The method of any one of claims 33-37, wherein the dispersing agent comprises a phosphoric acid polymer.

39. The method of claim 38, wherein the phosphoric acid polymer comprises carboxyl-PEG-phosphoric acid.

40. The method of any one of claims 25-39, wherein the substantially transparent magnetic polymer layer is deposited by spin coating or spray coating.

41. The method of any one of claims 25-40, wherein the deposited substantially transparent magnetic polymer layer is patterned by photolithography, lift-off, or sputtering.

42. The method of any one of claims 25-41, further comprising: coupling a capture agent for capturing an analyte to at least one of the first surface and the second surface of the substantially transparent magnetic polymer layer.

43. The method of claim 42, wherein coupling the capture agent comprises:
reacting the substantially transparent polymer of the substantially transparent magnetic polymer layer with a photoacid generator and light to generate a cross-linked polymer, wherein the light is of a wavelength that activates the photoacid generator;
reacting the epoxide of the cross-linked polymer with a compound comprising an amine and a carboxyl, wherein the amine of the compound reacts with the epoxide to form a compound-coupled, cross-linked polymer; and
reacting the carboxyl of the compound-coupled, cross-linked polymer with the capture agent to couple the capture agent to the substantially transparent magnetic polymer layer.

44. The method of any one of claims 25-43, wherein the magnetic nanoparticles are superparamagnetic.

45. The method of any one of claims 25-44, wherein the magnetic nanoparticles are less than about 30 nm in diameter and greater than or equal to about 3 nm in diameter.

46. The method of any one of claims 25-45, wherein the plurality of magnetic nanoparticles comprises less than about 10% (by weight) and greater than about 0.1% (by weight) of the mixture of the substantially transparent polymer and the plurality of magnetic nanoparticles.

47. The method of any one of claims 25-46, wherein the substantially transparent magnetic polymer layer is between about 0.1 μm and about 50 μm in thickness.

48. The method of any one of claims 25-47, wherein the substantially transparent polymer of the substantially transparent magnetic polymer layer is an epoxy-based polymer.

49. The method of claim 48, wherein the epoxy-based polymer is SU-8.

50. The method of any one of claims 25-49, wherein the substantially non-transparent layer comprises a substantially non-transparent polymer.

51. The method of claim 50, wherein the substantially non-transparent layer comprises a black matrix resist.

52. The method of claim 50, wherein the substantially non-transparent polymer exhibits an absorbance of greater than about 1.8 (OD) at a wavelength between about 230 nm and about 660 nm.

53. The method of any one of claims 25-49, wherein the substantially non-transparent layer comprises a metal that lacks residual magnetism.

54. The method of claim 53, wherein the substantially non-transparent layer comprises titanium or chromium.

55. The method of any one of claims 25-54, wherein the substantially non-transparent layer is between about 0.05 μm and about 2 μm in thickness.

56. The method of any one of claims 25-55, wherein the analog code comprises one or more overlapping or partially overlapping arc elements forming a continuous or discontinuous ring.

57. The method of any one of claims 25-56, wherein the deposited substantially non-transparent layer is patterned so as to provide an asymmetry.

58. The method of any one of claims 25-57, wherein the microcarrier is patterned in (b) into a substantially circular disc.

59. The method of claim 58, wherein the microcarrier is between about 5 μm and about 200 μm in diameter.

60. The method of claim 59, wherein the microcarrier is about 40 μm in diameter.

61. The method of any one of claims 25-60, wherein the microcarrier is less than about 50 μm in thickness.

62. The method of claim 61, wherein the microcarrier is between about 2 μm and about 10 μm in thickness.

63. The method of any one of claims 42-62, wherein the analyte is selected from the group consisting of a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment.

64. The method of any one of claims 42-63, wherein the capture agent for capturing the analyte is selected from the group consisting of a DNA molecule, a DNA-analog-molecule, an RNA-molecule, an RNA-analog-molecule, a polynucleotide, a protein, an enzyme, a lipid, a phospholipid, a carbohydrate moiety, a polysaccharide, an antigen, a virus, a cell, an antibody, a small molecule, a bacterial cell, a cellular organelle, and an antibody fragment.

65. An encoded micocarrier produced by the method of any one of claims 25-64.

66. A method for detecting multiple analytes in a solution, comprising:
(a) contacting a solution comprising a first analyte and a second analyte with a plurality of microcarriers, wherein the plurality of microcarriers comprises at least:
(i) a first microcarrier according to any one of claims 1-24 and 65 that specifically captures the first analyte, wherein the first microcarrier is encoded with a first analog code; and
(ii) a second microcarrier according to any one of claims 1-24 and 65 that specifically captures the second analyte, wherein the second microcarrier is encoded with a second analog code, and wherein the second analog code is different from the first analog code;
(b) decoding the first analog code and the second analog code using analog shape recognition to identify the first microcarrier and the second microcarrier; and
(c) detecting an amount of the first analyte bound to the first microcarrier and an amount of the second analyte bound to the second microcarrier.

67. The method of claim 66, wherein (b) occurs before (c).

68. The method of claim 66, wherein (c) occurs before (b).

69. The method of claim 66, wherein (b) and (c) occur simultaneously.

70. The method of any one of claims 66-69, wherein decoding the first analog code and the second analog code comprises:
(i) illuminating the first and second microcarriers by passing light through the substantially transparent magnetic polymer layers of the first and second microcarriers and/or the surrounding solution, wherein the light fails to pass through the substantially non-transparent layers of the first and second microcarriers to generate a first analog-coded light pattern corresponding to the first microcarrier and a second analog-coded light pattern corresponding to the second microcarrier;
(ii) imaging the first analog-coded light pattern to generate a first analog-coded image and imaging the second analog-coded light pattern to generate a second analog-coded image; and
(iii) using analog shape recognition to match the first analog-coded image with the first analog code and to match the second analog-coded image with the second analog code.

71. The method of any one of claims 66-70, wherein detecting the amount of the first analyte bound to the first microcarrier and the amount of the second analyte bound to the second microcarrier comprises:
(i) after (a), incubating the first and the second microcarriers with a detection agent, wherein the detection agent binds the first analyte captured by the first microcarrier and the second analyte captured by the second microcarrier; and
(ii) measuring the amount of detection agent bound to the first and the second microcarriers.

72. The method of claim 71, wherein the detection agent is a fluorescent detection agent, and wherein the amount of detection agent bound to the first and the second microcarriers is measured by fluorescence microscopy.

73. The method of claim 71, wherein the detection agent is a luminescent detection agent, and wherein the amount of detection agent bound to the first and the second microcarriers is measured by luminescence microscopy.

74. The method of any one of claims 66-73, wherein the solution comprises a biological sample.

75. The method of claim 74, wherein the biological sample is selected from the group consisting of blood, urine, sputum, bile, cerebrospinal fluid, interstitial fluid of skin or adipose tissue, saliva, tears, bronchial-alveolar lavage, oropharyngeal secretions, intestinal fluids, cervico-vaginal or uterine secretions, and seminal fluid.

76. A kit for conducting a multiplex assay comprising a plurality of microcarriers, wherein the plurality of microcarriers comprises at least:
(a) a first microcarrier according to any one of claims 1-24 and 65 that specifically captures a first analyte, wherein the first microcarrier is encoded with a first analog code; and
(b) a second microcarrier according to any one of claims 1-24 and 65 that specifically captures a second analyte, wherein the second microcarrier is encoded with a second analog code, and wherein the second analog code is different from the first analog code.

77. The kit of claim 76, further comprising a detection agent for detecting an amount of the first analyte bound to the first microcarrier and an amount of the second analyte bound to the second microcarrier.

78. The kit of claim 76 or claim 77, further comprising instructions for using the kit to detect the first analyte and the second analyte.
